(19) United States
(12) Patent Application Publication
(10) Pub. No.: US 2024/0290448 A1
(43) Pub. Date: Aug. 29, 2024

(54) SYSTEMS AND METHODS FOR LONGITUDINAL CARDIOLOGY TIMELINE PRESENTATION AND CLINICAL DECISION SUPPORT

(71) Applicant: GE Precision Healthcare LLC, Waukesha, WI (US)

(72) Inventors: Debra Umlauft, Slinger, WI (US); Attila Vojtek, Budapest (HU); Monica Masini, Aldershot (GB); Levente Árpád, Békéscsaba (HU); Paul Rose, Seattle, WA (US); Péter Benis, Tárnok (HU)

(21) Appl. No.: 18/589,242

(22) Filed: Feb. 27, 2024

Related U.S. Application Data
(60) Provisional application No. 63/487,562, filed on Feb. 28, 2023.

(51) Int. Cl. G16H 10/60; G06F 3/0482; G06F 3/0484; G16H 50/20

(52) U.S. Cl. CPC G16H 10/60; G06F 3/0482; G06F 3/0484; G16H 50/20

(57) ABSTRACT

Various methods and systems are provided for a longitudinal cardiology patient history timeline and clinical decision support system. In one example, a computing device comprising a display screen displays a menu listing one or more electronic medical records (EMRs) of one or more patients, and additionally is configured to display a patient timeline graphical user interface (GUI) accessible from the menu while the one or more EMR systems are in an un-launched state. The patient timeline GUI displays patient data as longitudinal medical history event elements. The computing device is additionally configured to generate one or more activities based on the patient data. The patient data is obtained from the one or more EMRs and the medical history event elements are selectable to launch a pop-up window with additional information relating to the selected event.

### CROSS-REFERENCE TO RELATED APPLICATIONS

The present application claims priority to U.S. Provisional Application No. 63/487,562, entitled “SYSTEMS AND METHODS FOR LONGITUDINAL CARDIOLOGY TIMELINE PRESENTATION AND CLINICAL DECISION SUPPORT”, and filed on Feb. 28, 2023. The entire contents of the above-listed application are hereby incorporated by reference for all purposes.

### FIELD

Embodiments of the subject matter disclosed herein relate to care guideline recommendations, and more particularly to an integrated cardiology timeline presentation system including clinical decision support.

### BACKGROUND

Digital collection, processing, storage, and retrieval of patient medical records may include a conglomeration of large quantities of data. In some examples, the data may include numerous medical procedures and records generated during investigations of the patient, including a variety of examinations, such as blood tests, urine tests, pathology reports, image-based scans, etc. Duration of the diagnosis of a medical condition of a subject followed by treatment may be spread over time from a few days to a few months or even years in the case of chronic diseases, including cardiac or cardiology-related conditions, which may be diseases that take more than one year to cure/treat or in some instances may be lifelong. Over the course of diagnosing and treating chronic disease, the patient may undergo many different treatments and procedures and may move to different hospitals and/or geographic locations.

Physicians are increasingly relying on electronic medical record (EMR) systems to go through historical health records of the patient during diagnosis, treatment, and monitoring of patient conditions. For patients with chronic cardiac conditions, hundreds or even thousands of EMRs may result from numerous visits. Information may also be included in various other data sources, such as radiology systems, picture archiving and communication systems, and many more. Sorting and extracting information from multiple data sources is slow and inefficient, increasing likelihood of missing records when determining care or treatment plans due to data being spread out across a large number of records.

### BRIEF DESCRIPTION

In one example, a computing device comprising a display screen, the computing device being configured to display on the display screen a menu listing one or more electronic medical record (EMRs) of one or more patients, and additionally being configured to display on the display screen a patient timeline graphical user interface (GUI) accessible from the menu. The patient timeline GUI may display, for each patient, patient data as longitudinal medical history event elements. The patient data may be obtained from the one or more EMRs, wherein each element of the longitudinal medical history event elements is selectable to launch a pop-up window with additional information relating to the selected element. The computing device is additionally configured to generate one or more activities based on the patient data. The patient timeline GUI may be displayed while the one or more EMRs are in an un-launched state.

In another example, a method for a longitudinal cardiology timeline and clinical decision support system comprises displaying a menu listing one or more options for retrieving data of one or more patients from a plurality of data repositories of a hospital, the plurality of data repositories including one or more electronic medical record (EMR) systems; displaying a patient timeline graphical user interface (GUI) that displays, for each patient, a plurality of elements indicating a plurality of history events determined from the retrieved data from the one or more EMR systems, the plurality of elements being arranged chronologically; and in response to selection of an element of the plurality of elements, modifying the patient timeline GUI to display a clinical decision support GUI that displays one or more activity items, wherein the patient timeline GUI is displayed while the one or more EMR systems are in an un-launched state.

In another example, a longitudinal cardiology patient history timeline system, comprises one or more processors; and memory storing instructions executable by the one or more processors to: output, for display on a display device, a patient timeline graphical user interface (GUI) that includes, for a patient, a plurality of panels indicating patient history events, where each panel is generated by applying a set of rules to a set of patient data obtained from an electronic medical record database; and display, within a modification of the patient timeline GUI, an activity recommendation indicating a suggested care measure based on the patient data.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows a block diagram of an example system for displaying cardiology-focused clinical information of a patient to a user and generating clinical decision support;

FIG. 2 shows an example patient timeline graphical user interface (GUI) generated with the system of FIG. 1;

FIG. 3 shows an example clinical decision support GUI;

FIG. 4 shows an example patient timeline GUI with a clinical decision support GUI;

FIG. 5 shows an example patient timeline GUI with a pop-up window;

FIG. 6 shows another example patient timeline GUI;

FIG. 7 shows another example clinical decision support GUI;

FIG. 8 shows another example clinical decision support GUI;

FIG. 9 shows another example clinical decision support GUI;

FIG. 10 shows an example add activity pop-up window;

FIG. 11 shows an example algorithmic score pop-up window;

FIG. 12 shows a flowchart illustrating a method for generating and displaying a timeline and one or more activity items for a patient;

FIG. 13 shows a flowchart illustrating a method for display of clinical decision support via one or more activity items;

FIG. 14 shows a flowchart illustrating a method for identifying activities for display within a clinical decision support GUI; and

FIG. 15 shows another example of a patient timeline GUI.

### DETAILED DESCRIPTION

The following description relates to various embodiments of a longitudinal cardiology patient history timeline and clinical decision support system. In particular, systems and methods for a longitudinal cardiology patient history timeline and clinical decision support system are provided for patient history analysis and display of longitudinal patient information that structures a patient's medical data into a visual longitudinal patient journey view as well as display of decision support that provides recommendation(s) for treatment and/or care to aid clinical thinking and guide actions to achieve efficiency and personalized patient experience.

Hospitals and other clinical facilities may provide computing systems with graphical user interfaces (GUIs) for displaying patient information to healthcare providers and other users. In this way, a healthcare provider may view historical and the most up-to-date patient information and retrieve data from electronic medical records (EMRs), imaging results, laboratory results, and so on. Further, alerts may be automatically and/or manually generated to indicate recommendations and/or guidelines for care of a patient. Such alerts are often retrieved from a single source (e.g., a single EMR system, a picture archiving and communication system (PACS), etc.). Retrieving information in this manner may result in display of only one data type or medical modality, and therefore falls short of comprehensive patient-centric analysis. For example, patient information retrieved from an EMR may exclude information of recently updated imaging. As such, care recommendations may not take into account results of the recently updated imaging and therefore are not comprehensive and recommendations for clinical decisions or treatment plans may be inaccurate and/or may not maximize patient outcomes.

Cardiovascular disease is a common chronic condition that demands long term monitoring, treatment, and intervention, often over many years of a patient's life, therefore resulting in numerous encounters, exams, lab draws, etc. Cardiovascular disease may include a variety of conditions including heart disease (coronary artery disease), heart attacks (myocardial infarctions), stroke (cerebrovascular accident), heart failure, arrhythmias, valvular disease, peripheral vascular disease, and more. Clinical and other medical data, such as imaging findings, laboratory results, vital signs, including blood pressure and heart rate, algorithmic scores, and heart rhythm findings, among others, are used to diagnose and monitor cardiovascular health of a patient. With long term monitoring and treatment of such a chronic disease, the medical data of a patient's past medical history may become vast and difficult to sort through when searching for specific information when making informed clinical decisions. As a result, cognitive overload and suboptimal care due to missing information may occur.

The methods and systems provided herein detail a longitudinal cardiology patient history timeline and clinical decision support system. The patient history timeline may consolidate multiple types of medical data from a plurality of sources (e.g., a plurality of data repositories) into a single graphical user interface that enables users to easily review a patient's medical history, visualize and evaluate a specialty-centric (e.g., cardiology-centric) health journey, review response to various treatments and/interventions, and formulate future decisions about a patient's care. The system further analyzes the medical data to generate one or more activity items based on known care guidelines. The user may accept, reject, or propose alternative activities within a clinical decision support interface and may export a resulting care plan for their own use as well as for patient use.

Via user interactions with graphical user interfaces, the user (e.g., the care provider) may view patient data in various forms, including in brief small text as well as in detailed views via user selection of elements (e.g., hovering over an element to launch a pop-up window). Further, user interaction may designate which of the recommended activities are to be included in a care plan for a patient. The recommended activities may guide the care provider in decision making, thereby reducing cognitive overload and providing increased efficiency. Further, the systems and methods herein described may reduce demanded processing power and increase efficiency of the computing system.

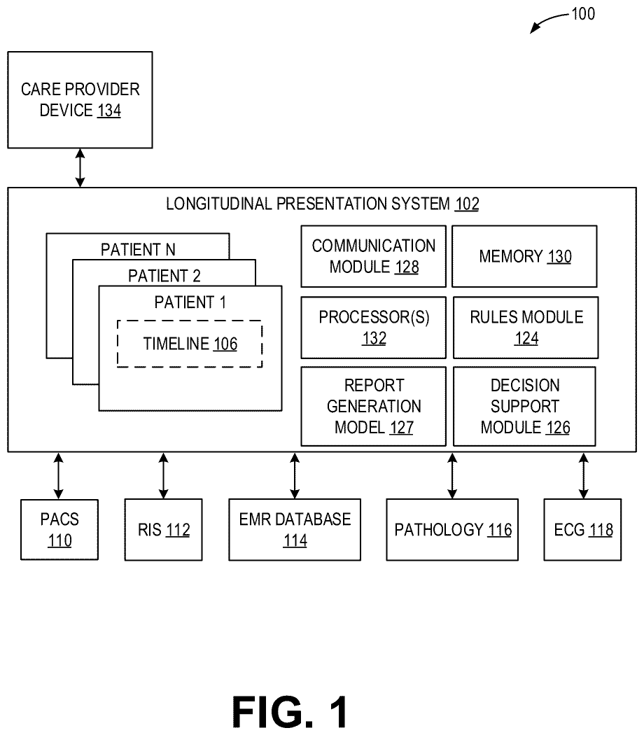

Embodiments of the present disclosure will now be described, by way of example, with reference to the figures, in which FIG. 1 schematically shows an example patient information system 100 that may be implemented in medical facility such as a hospital. Patient information system 100 may be a portion of or otherwise included in a computing device and/or computing system. Patient information system 100 may include a longitudinal presentation system 102. Presentation system 102 may include resources (e.g., memory 130, processor(s) 132) that may be allocated to store and execute timelines for each of a plurality of patients. For example, as shown in FIG. 1, timeline 106 is stored on presentation system 102 for a first patient (patient 1); a plurality of additional timelines may be stored on and/or generated by presentation system 102, each corresponding to a respective patient (patient 2 up to patient N).

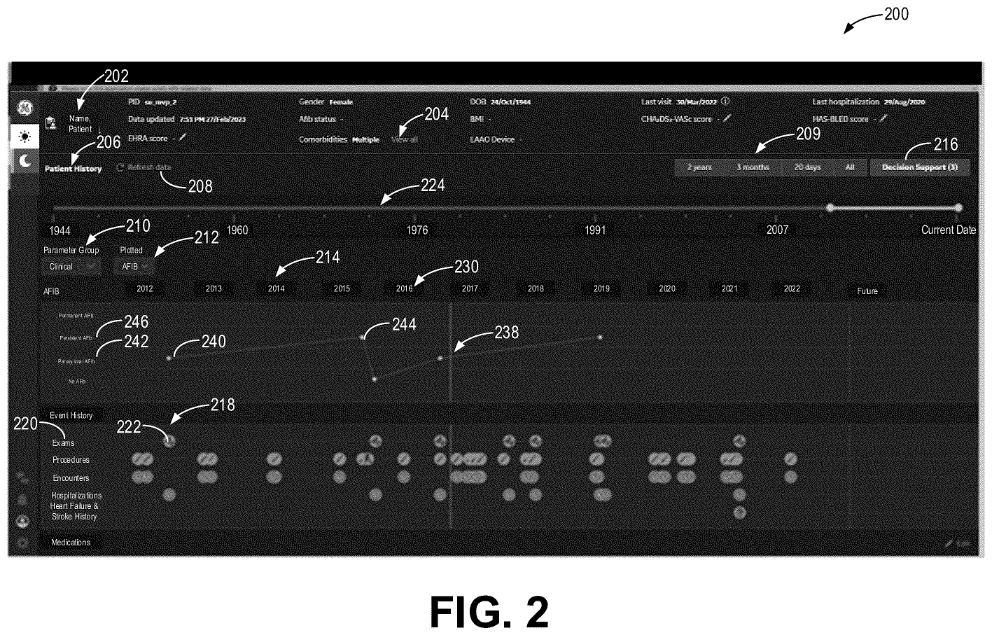

Each timeline 106 may include graphical representations of patient medical events arranged chronologically, as will be described with reference to FIG. 2. The patient medical events depicted on the timeline 106 may include office or hospital visits (and information gathered during such visits, including vital signs and point of care lab results), findings from diagnostic imaging, pathology reports, lab test results, algorithmic scores, and any other clinically relevant information. Further, the patient medical information, including medical history, current state, vital signs, and other information, may be analyzed by a decision support module 126, which may be used to generate and output recommendations for care and/or treatment.

The patient information that is presented via the timeline 106 may be stored in different medical databases or storage systems (e.g., data repositories) in communication with presentation system 102. For example, as shown, the presentation system 102 may be in communication with a PACS 110, a radiology information system (RIS) 112, an EMR database 114, a pathology database 116, and an electrocardiogram (ECG) management system 118, and/or other data sources such as a clinical information system (CIS), hospital information system (HIS), or others. PACS 110 may store medical images and associated reports (e.g., clinician findings), such as ultrasound images, MRI images, etc. PACS 110 may store images and communicate according to digital imaging and communications in medicine (DICOM) format. RIS 112 may store radiology images and associated reports, such as computerized tomography (CT) images, X-ray images, etc. EMR database 114 store electronic medical records for a plurality of patients. EMR database 114 may be a database stored in a mass storage device configured to communicate with secure channels (e.g., HTTPS and TLS), and store data in encrypted form. Further, the EMR database is configured to control access to patient electronic medical records such that only authorized healthcare providers may edit and access the electronic medical records. An EMR for a patient may include patient demographic information, family medical history, past medical history, lifestyle information, preexisting medical conditions, current medications, allergies, surgical history, past medical screenings and procedures, reports from past hospitalization, including discharge summaries, progress notes, and the like, notes from outpatient visits, etc. Pathology database 116 may store pathology images and related reports, which may include visible light or fluorescence images of tissue, such as immunohistochemistry (IHC) images. ECG management system 118 may store data of ECG tracings, including results of ECGs (e.g., heart rate, rhythm, parameters like QT interval and QRS complex).

When requested, timeline 106 may be displayed on the one or more display devices. As shown in FIG. 1, a care provider device 134, and in some examples more than one care provider device, may be communicatively coupled to presentation system 102. Each care provider device may include a processor, memory, communication module, user input device, display (e.g., screen or monitor), and/or other subsystems and may be in the form of a desktop computing device, a laptop computing device, a tablet, a smart phone, or other device. Each care provider device may be adapted to send and receive encrypted data and display medical information, including medical images in a suitable format such as DICOM or other standards. The care provider devices may be located locally at the medical facility (such as in a room of a patient or a clinician's office) and/or remotely from the medical facility (such as a care provider's mobile device).

When viewing timeline 106 via a display of a care provider device, a care provider may enter input (e.g., via the user input device, which may include a keyboard, mouse, microphone, touch screen, stylus, or other device) that may be processed by the care provider device and sent to the presentation system 102. In examples where the user input is a selection of a link or user interface control button/element of the timeline, the user input may trigger display of a selected EMR, trigger progression to a desired point in time or view of the timeline (e.g., trigger display of desired patient medical information), trigger display of various conditions in the timeline or display of information specific to a condition or event, trigger updates to the configuration of the timeline, trigger modification of the patient timeline GUI with display of a clinical decision support GUI, or other actions.

In some examples, the presentation system 102 may include the decision support module 126 that may be configured to analyze patient data obtained from the plurality of sources (e.g., EMR database 114, PACS 110, RIS 112, pathology 116, and/or ECG management system 118). The patient data may include patient history events and information relevant to those events, including dates of evaluation and findings for imaging data, laboratory results, vital signs, pathology, ECG data, hospitalization records, and more. The decision support module 126 may be in communication with a rules module 124. The rules module 124 may include rule sets and criteria for a plurality of guideline recommendations. In some examples, guideline recommendations may be sourced from a widely available (e.g., publically published) source (e.g., guidelines from American College of Cardiology (ACC) and/or European Society of Cardiology (ESC)) and may be storied in memory 130 of the patient information system 100. In other examples, guideline recommendations may be customizable for configuration by a user (e.g., a clinical expert or authorized care provider).

Criteria for a guideline recommendation may include one or more triggers (e.g., patient data acquired from the plurality of sources) that may be met in order for the guideline recommendation to be indicated. Each trigger may be defined based on a procedure code stored in the memory 130 and thus determination of whether a particular trigger has been met may be based on a procedure code within patient information (e.g., an EMR) matching a procedure code stored in memory as part of the criteria for guideline recommendations. A procedure code may define what was done or administered to a patient in a clinical setting, this may be a surgical procedure, a screening test, a diagnostic exam, a medication, a diagnosis given to the patient, an algorithmic score defined for the patient, among others. Codes are alphanumeric and allow for identification of terminology in data repositories to identify triggers. Types of terminologies include International Classification of Diseases (ICD)-10 codes, Current Procedural Terminology (CPT) codes, RxNorm, and the like. In some examples, the procedure code that defines (e.g., identifies) triggers, which is stored in the memory 130, may be determined automatically by the patient information system 100. In other examples, the procedure code that defines triggers may be configured manually by a clinical expert or care provider and inputted into the system for storage in memory 130. Further, in some examples, a user may configure from which source guideline recommendations are taken, for example either the ACC or the ESC, if a care region is specified or user preference is had.

Each guideline recommendation may include or otherwise prescribe one or more activities. Activities may be treatments, interventions, or other types of care that may be prescribed to a patient. When recommended based on an indicated guideline recommendation, an activity may be displayed as an activity item (e.g., an activity recommendation) to the user via a clinical decision support interface. Rule sets may include trigger combinations that may be met in order for an activity of an indicated guideline recommendation to be suggested for a specified patient. Each rule set may include a decision tree that includes parameter connectors “AND”, “OR”, and/or “AND/OR” that define relationships between parameters. Combinations with AND must include each parameter connected with the AND. Combinations with OR may include any of the parameters connected with the OR. Combinations with AND/OR may include one or both (or all, for combinations including more than two parameters) parameters connected with the AND/OR. Trigger combination strings may include multiple connections, wherein each parameter is connected to at least one other parameter via a parameter connector.

As a non-limiting example, for an activity suggesting opportunistic screening for atrial fibrillation, a first parameter may be no historical diagnosis of atrial fibrillation, a second parameter may be arterial hypertension, and a third parameter may be no ECG within 180 days. A decision tree of a rule set corresponding to the activity may stipulate that the first parameter AND the second parameter AND the third parameter must be met in order for the activity to be identified. Patient data that includes no history of atrial fibrillation, a current diagnosis of hypertension, and a most recent ECG dated 2 years previous may satisfy the rule set and the corresponding activity may be displayed as an activity item for the patient. Conversely, patient data that includes no history of atrial fibrillation and a current diagnosis of hypertension, but a most recent ECG is dated 15 days previous may not satisfy the rule set as the third parameter is not met and therefore the corresponding activity may not be displayed as an activity item for the patient. Satisfaction of the rule set may be determined based on the decision tree therein, whereby a decision tree algorithm and matching of procedure codes determines whether parameters/triggers are met by patient data. In this way, only relevant recommendations are displayed for a patient of interest.

Determination of activities may be performed by a decision tree algorithm of the decision support module 126 based on the defined rule sets or based on another suitable mechanism. Each decision tree and rule set may be stored in memory 130 and accessed to perform an associated decision tree algorithm. Decision trees and the decision tree algorithms used to determine activities may also be configurable, in some examples. As an example, the system may allow a user to add, modify, or delete one or more decision trees or algorithms used to generate activity recommendations.

The rules module 124 may further include period segmentation rules. The period segmentation rules may arrange the patient data chronologically and group (e.g., segment) patient data into a plurality of events. For example, medical data obtained from the plurality of sources for a specified patient may include data of a plurality of events such as hospitalizations, encounters, exams (e.g., imaging exams or other), disease events (e.g., strokes, heart attacks, heart failure exacerbations, new arrhythmias, among others), medication changes (e.g., starts, stops, or dosage changes), and more. Each of the plurality of events may include one or more data points at various points in time. The period segmentation rules may group the patient data into the plurality of events and arrange each data point for each event chronologically. In this way, different events may be displayed within separate time aligned panels and/or graphs of a corresponding user interface in a way that organizes the patient data to be easily visualized by the care provider, thereby increasing efficiency and decreasing cognitive overload.

In some examples, the presentation system 102 may include a report generation model 127 that may be configured to generate patient-customized report templates based on accepted activity items from within the clinical decision support GUI. The report generation model 127 may include one or more rule sets for each activity to generate information relevant to each activity accepted for a specified patient. For example, in an example in which an activity item is a medication change, the report generation model 127 may generate information related to prior prescription, new prescription (including information of dosage, frequency, number of pills prescribed, etc.), instructions for use, and more, which may be included in a generated report of a care plan.

A management application executed of the presentation system 102 may allow an administrator to configure how the timelines are displayed, what information is conveyed by the timelines for each patient, what is included in each rule set, etc. The management application may include an interface for configuring hospital specific protocols and guidelines for generating and displaying the timelines. The management application may further allow the administrator, clinical expert, or authorized care provider to modify criteria and/or rule sets for guideline recommendations and/or activities. In some examples, modifications made to criteria and/or rule sets may be personalized to a specific care provider. In other examples, modifications to criteria and/or rule sets may be made for a medical facility, network, or other entity in which changes made apply to each user within the entity.

Presentation system 102 may further include a communication module 128, memory 130, and processor(s) 132 to store and generate the timelines, as well as send and receive communications, GUIs, medical data, and other information.

Communication module 128 facilitates transmission of electronic data within and/or among one or more systems. Communication via communication module 128 can be implemented using one or more protocols. In some examples, communication via communication module 128 occurs according to one or more standards (e.g., DICOM, Health Level Seven (HL7), ANSI X12N, etc.). Communication module 128 can be a wired interface (e.g., a data bus, a Universal Serial Bus (USB) connection, etc.) and/or a wireless interface (e.g., radio frequency, infrared, near field communication (NFC), etc.). For example, communication module 128 may communicate via wired local area network (LAN), wireless LAN, wide area network (WAN), etc. using any past, present, or future communication protocol (e.g., BLUETOOTH™, USB 2.0, USB 3.0, etc.).

Memory 130 may include one or more data storage structures, such as optical memory devices, magnetic memory devices, or solid-state memory devices, for storing programs and routines executed by processor(s) 132 to carry out various functionalities disclosed herein. Memory 130 may include any desired type of volatile and/or non-volatile memory such as, for example, static random access memory (SRAM), dynamic random access memory (DRAM), flash memory, read-only memory (ROM), etc. Processor(s) 132 may be any suitable processor, processing unit, or microprocessor, for example. Processor(s) 132 may be a multi-processor system, and, thus, may include one or more additional processors that are identical or similar to each other and that are communicatively coupled via an interconnection bus. As an example, the decision support module 126 may store instructions for generating activity items in the memory 130 that are executable by the processor(s) 132.

As used herein, the terms “sensor,” “system,” “unit,” or “module” may include a hardware and/or software system that operates to perform one or more functions. For example, a sensor, module, unit, or system may include a computer processor, controller, or other logic-based device that performs operations based on instructions stored on a tangible and non-transitory computer readable storage medium, such as a computer memory. Alternatively, a sensor, module, unit, or system may include a hard-wired device that performs operations based on hard-wired logic of the device. Various modules or units shown in the attached figures may represent the hardware that operates based on software or hardwired instructions, the software that directs hardware to perform the operations, or a combination thereof.

“Systems,” “units,” “sensors,” or “modules” may include or represent hardware and associated instructions (e.g., software stored on a tangible and non-transitory computer readable storage medium, such as a computer hard drive, ROM, RAM, or the like) that perform one or more operations described herein. The hardware may include electronic circuits that include and/or are connected to one or more logic-based devices, such as microprocessors, processors, controllers, or the like. These devices may be off-the-shelf devices that are appropriately programmed or instructed to perform operations described herein from the instructions described above. Additionally, or alternatively, one or more of these devices may be hard-wired with logic circuits to perform these operations.

Thus, presentation system 102 may be configured to obtain/ingest medical data from a variety of sources (e.g., PACS, EMR, RIS, etc.) and analyze, extract, and register selected medical data to generate a timeline and activity item(s) for each patient as described herein. In some examples, presentation system 102 may include one or more data filters (e.g., AI-assisted data filters) configured to monitor and filter the ingested data to ensure that only relevant and complete data is presented in the timeline. In some examples, an indication of the level of confidence in the data may be presented with an icon in each timeline. This adds to the confidence factors in a clinical solution and guideline recommendations and also leans towards being representative of precision health. This would apply to quality checks on imaging data and IQ evaluation of digital pathology, radiology (ensuring appropriateness of protocols for the condition adjudged).

One or more of the devices described herein may be implemented over a cloud or other computer network. For example, presentation system 102 is shown in FIG. 1 as constituting a single entity, but it is to be understood that presentation system 102 may be distributed across multiple devices, such as across multiple servers. Further, while the elements of FIG. 1 are shown as being housed at a single medical facility, it is to be appreciated that any of the components described herein (e.g., EMR database, RIS, PACS, etc.) may be located off-site or remote from the presentation system 102. Further, the longitudinal data utilized by the presentation system 102 for the timeline generation and other tasks described below could come from systems within the medical facility or obtained through electronic means (e.g., over a network) from other referring institutions.

While not specifically shown in FIG. 1, additional devices described herein (e.g., care provider device 134) may likewise include user input devices, memory, processors, and communication modules/interfaces similar to communication module 128, memory 130, and processor(s) 132 described above, and thus the description of communication module 128, memory 130, and processor(s) 132 likewise applies to the other devices described herein. As an example, the care provider devices (e.g., care provider device 134) may store user interface templates in memory that include placeholders for relevant information stored on presentation system 102 or sent via presentation system 102. For example, care provider device 134 may store a user interface template for a patient timeline that a user of care provider device 134 may configure with placeholders for desired patient information. When the timeline is displayed on the care provider device, the relevant patient information may be retrieved from presentation system 102 and inserted in the placeholders. The user input devices may include keyboards, mice, touch screens, microphones, or other suitable devices.

Turning now to FIG. 2, an example of a patient timeline GUI 200 is shown that may be generated for a patient by presentation system 102. Patient timeline GUI 200 may be displayed on a display of a care provider device (e.g., care provider device 134 of FIG. 1). Patient timeline GUI 200 may include a plurality of different categories of timelines that may be displayed together or individually in a time-aligned manner as different panels of the patient timeline GUI 200. Data points and icons within each of the different panels may indicate or represent a plurality of history events and patient parameters retrieved from a plurality of data repositories (e.g., EMRs, PACS, etc.). Patient timeline GUI 200, and other GUIs herein described, may be outputted to the display device in response to user selection of a selectable link within one or more EMRs or may be a standalone application accessible by the computing device. The presentation system 102 by which information displayed within the patient timeline GUI 200 is generated may be patient centric, such that a link within an EMR may be specific to a patient and selection of the link may trigger display of information within the patient timeline GUI 200 relevant to that patient. It should be understood that while the patient timeline GUI 200, and other patient timeline GUIs herein, is described with respect to cardiology-centric data, other types of medical data may be displayed in a similar fashion without departing from the scope of this disclosure.

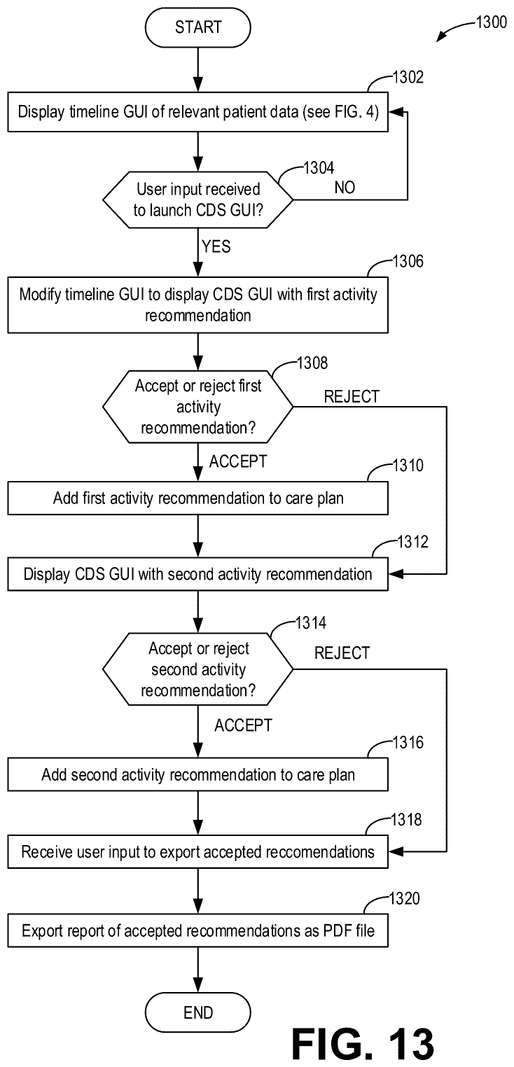

Patient timeline GUI 200 may include a patient information panel 202 that includes 1) a plurality of patient demographics including patient name, gender, date of birth, body mass index (BMI), last hospitalization, among others; 2) a plurality of cardiology-specific data, including atrial fibrillation status, a congestive heart failure-hypertension-age-diabetes-stroke-vascular disease (CHA₂DS₂-VASc) score, and a hypertension-abnormal renal/liver function-stroke-bleeding history or predisposition-labile international normalized ratio (INR)-elderly-drugs/alcohol concomitantly (HAS-BLED) score, among others; and/or 3) a date/time in which the data presented in the patient timeline GUI 200 was last updated. The patient information panel 202 may also include one or more selectable links 204 that when selected allow a user to input information regarding one or more of the data displayed in the patient information panel 202. In some examples, each of the one or more selectable links 204 corresponds to one of the presented demographics or cardiology-specific data (or other datum included in the patient information panel 202). For example, a selectable link may correspond to a HAS-BLED score that allows the user to input a determined score, as will be described further with respect to FIG. 13.

The patient timeline GUI 200 may further include a patient history panel 206. The patient history panel 206 may include a patient history timeline 224, a time aligned graph 214, and an event history panel 218. The patient history timeline 224 may indicate, from left to right, dates of available data for the patient. The user may select via user input a range of years/dates on the patient history timeline 224 that is to be presented in the patient timeline GUI 200. Alternative to selecting a range of years/dates on the patient history timeline 224, the user may select one of a plurality of predefined ranges of dates 209 from which data may be pulled by the presentation system 102. Additionally, the patient history panel may include a refresh element 208 that when selected triggers resampling of patient data to update information displayed in the patient timeline GUI 200.

A parameter group menu 210 may be further included in the patient history panel 206 that allows the user to select what parameters are to be displayed in the time aligned graph 214 from a drop-down menu. For example, a clinical parameter group may be selected in order to display clinical parameters (e.g., diagnoses, conditions, encounters, etc.). Other parameter groups that may be selected include but are not limited to lab, ECG, and algorithmic scores. Each parameter group may include one or more parameter types that may be plotted on a time aligned graph. For example, an ECG parameter group may include a plurality of parameters found in an ECG, such as a QT interval, heart rate, etc.

A plotted menu 212 may be further included in the patient history panel 206 that allows the user to select which condition, lab value, vital sign, etc., is plotted on the time aligned graph 214. Options available in a drop-down menu of the plotted menu 212 may be determined/filtered based on which parameter group is selected in the parameter group menu 210. For example, for the clinical parameter group, options displayed in the plotted menu 212 may include conditions such as atrial fibrillation, strokes, cardiovascular events such as myocardial infarctions, and/or the like, but may exclude lab values, which may be options for a lab parameter group.

The time aligned graph 214 may display a condition, laboratory test, algorithmic score, or other as based on the selected parameter from the plotted menu 212. The time aligned graph 214 may display one or more data points 238 in a chronological manner, each data point corresponding to a documented event relating to the parameter plotted. The parameter plotted on the time aligned graph 214 may include one or more headings and each data point included in the graph may correspond to one of the one or more headings. For example, for atrial fibrillation, the one or more headings of the time aligned graph 214 may be types of atrial fibrillation (e.g., permanent atrial fibrillation, persistent atrial fibrillation, paroxysmal atrial fibrillation, and no atrial fibrillation). Each of the data points may be subcategorized by the heading to which it corresponds. For example, a first data point 240 may correspond to a first heading 242 (e.g., paroxysmal atrial fibrillation) for a first date while a second data point 244 may correspond to a second heading 246 (e.g., persistent atrial fibrillation) for a second date.

In some examples, a timeline 230 of the time aligned graph 214 may include a range of dates specified by the patient history timeline 224 or by the plurality of predefined ranges of dates 209. The timeline 230 may be an abscissa of the time aligned graph 214 and the one or more headings may act as an ordinate of the graph.

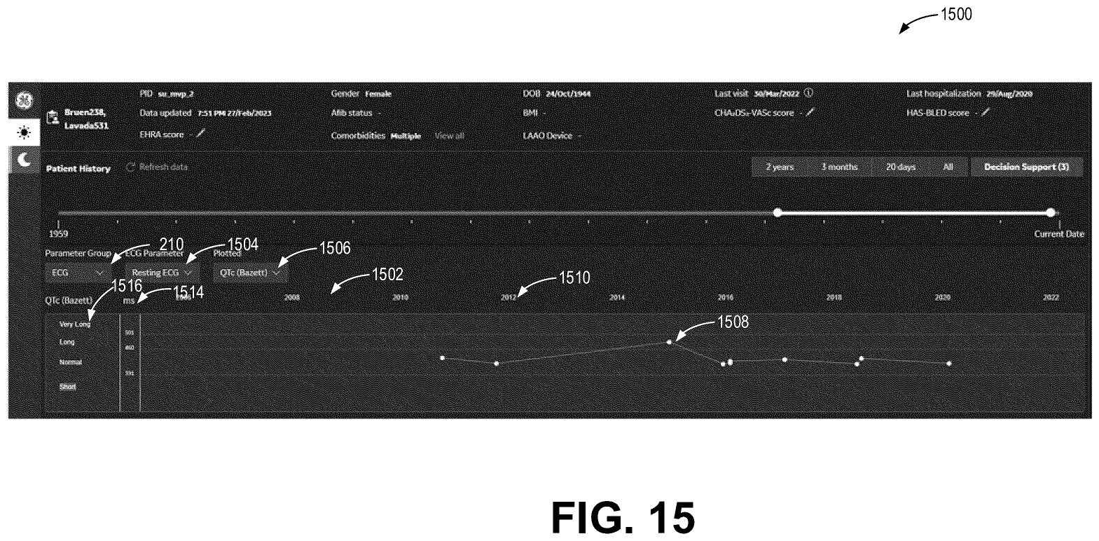

Referring briefly to FIG. 15, another example of a patient timeline GUI 1500 with a time aligned graph 1502 depicting a different plotted parameter is shown. In some examples, the patient timeline GUI 1500 may be the same patient timeline GUI as patient timeline GUI 200 of FIG. 2, with a different parameter plotted in the time aligned graph. While in FIG. 2, clinical is selected from the parameter group menu 210 and atrial fibrillation is plotted on the time aligned graph 214, in FIG. 15, ECG is selected from the parameter group menu 210, an ECG parameter type 1504 (e.g., resting ECG) is selected, and an ECG parameter 1506 (e.g., QTc) is plotted on the time aligned graph 1502. In some examples, the parameter type drop down menu and plotted drop down menu that are displayed within the patient timeline GUI may be based on which parameter group is selected from the parameter group menu 210.

Similar to the time aligned graph 214 as described with respect to FIG. 2, a plurality of data points 1508 are included in the time aligned graph 1502, displayed in a chronological manner. Each of the plurality of data points 1508 corresponds to a heading of one or more headings 1516 and to a date on a timeline 1510 of the time aligned graph 1502. The one or more headings 1516 may act as an ordinate of the graph and the timeline 1510 may act as an abscissa of the graph. For the plotted ECG parameter (e.g., QTc), the headings may indicate a category of QT interval (e.g., very long, long, normal, and/or short), similar to the types of atrial fibrillation described above.

The parameter group menu 210 may be included in the patient timeline GUI 1500 and the patient timeline GUI 200 when the patient timeline GUI 1500 is the same as the patient timeline GUI 200, with both clinical and ECG displayed in the drop-down menu therein. In contrast to the time aligned graph 214 of the patient timeline GUI 200, the time aligned graph 1502 also includes a units heading 1514. Values of the units heading 1514 may be specific to the categories shown in the one or more headings and may act as another ordinate for the plurality of data points 1508. In this way, user selection of parameter groups, parameter types, and parameters may affect display of the time aligned graph included in a patient timeline GUI, wherein headings and/or ordinates are specific to parameter selections.

Returning to FIG. 2, the event history panel 218 may also be time aligned according to the timeline 230. The event history panel 218 may include a plurality of types of events 220 including exams (e.g., non-invasive diagnostic exams), procedures, encounters, hospitalizations, disease/condition events (e.g., heart failure and stroke history), and/or other types of events. Each of the types of events 220 may include one or more event icons 222. Each of the event icons 222 may correspond to a date on the timeline 230.

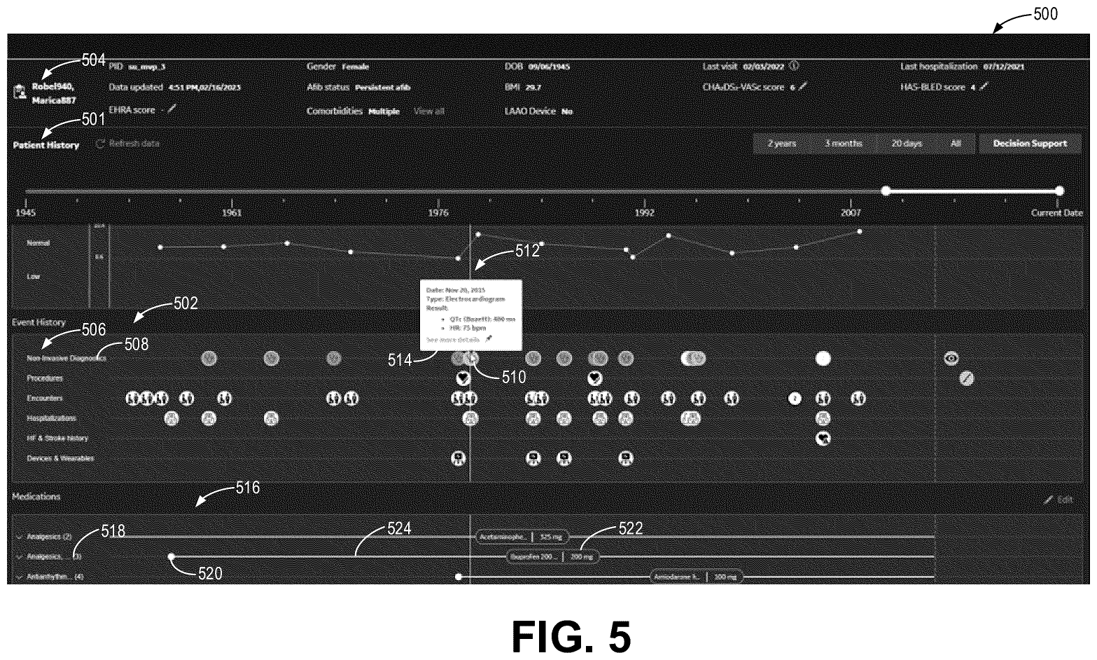

In some examples, each of the event icons 222 of the event history panel 218 and the one or more data points 238 of the time aligned graph 214 may be selectable. When selected or hovered over, additional information regarding the event which a selected event icon represents may be displayed in a pop-up window, as will be further described with respect to FIG. 5.

While not shown in FIG. 2, in some examples, the patient timeline GUI 200 may include additional panels, including but not limited to a response to treatment visualization and a medication panel. The response to treatment visualization may include a panel showing, for example, a diagram or silhouette of a heart with treated vessels (e.g., vessels treated during a coronary artery bypass graft procedure) shown as circles. Alternatively, or additionally, response to treatment visualization may include textual information regarding changes in cardiac health status (e.g., in the form of cardiac risk scores or other statuses) prior to and following various treatments and/or interventions, including procedural, surgical, or medical (e.g., medication management). The medication panel, as will be described further with respect to FIG. 5, may include relevant medication categories as headings and medication icons for each of the headings displayed with respect to time similar to the event history panel 218.

The patient timeline GUI 200 may further include a decision support element 216. The decision support element 216, when selected, may trigger a decision support module (e.g., decision support module 126 of FIG. 1) to generate or identify one or more activity items for the patient and may launch a clinical decision support GUI. The clinical decision support GUI may be a modification of the patient timeline GUI 200 whereby the clinical decision support GUI is displayed as a pop-up interface window or a side panel of the patient timeline GUI 200.

Thus, via patient timeline GUI 200, patient information relevant to the patient's condition (e.g., cardiology specific/cardiovascular condition) may be displayed in a time-ordered fashion. The patient information may be displayed via small graphical elements with minimal text, which may allow a large number of events, records, and reports to be included in the same timeline or graph. The user may select a graphical element of interest to view more information about the corresponding event, record, or report. The patient information may be stored in different databases that would otherwise be accessed via individual interfaces, and thus by aggregating the patient information via the patient timeline GUI 200, the amount of time necessary to review relevant patient information for diagnosis and treatment decisions, including time spent in review of guideline recommendations presented in a clinical decision support GUI, may be reduced.

The timelines disclosed herein aggregate patient data into a single place, e.g., into a single application, which helps reduce wasted time searching for known but scattered data, and unknown and missing data. The timelines reduce cognitive overloads and aid clinical thinking because the patient record data is reconstructed into a clinically helpful structure (co-morbidities complicates decision making) Care providers treating transfer patients or new patients can quickly get to diagnosis or treatment completion if such a simple multi-omic view is shown.

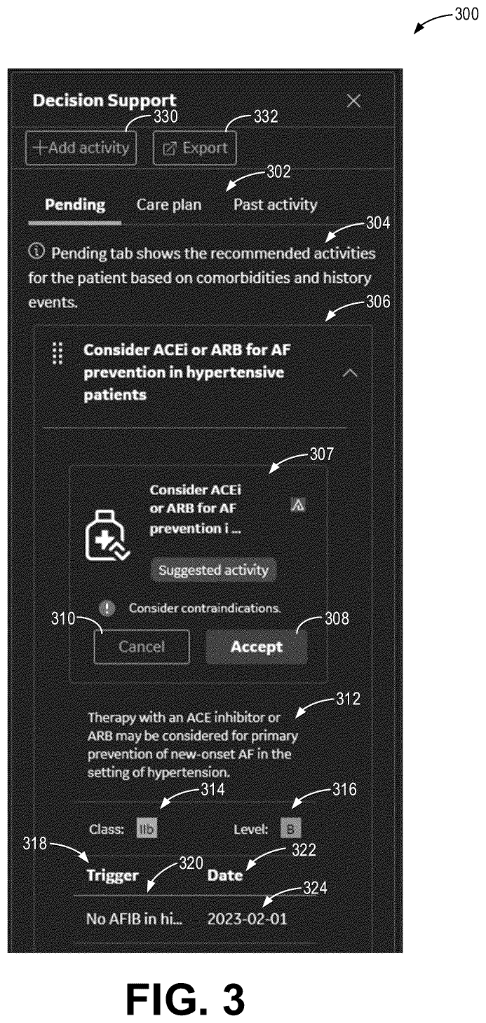

Turning now to FIG. 3, an example clinical decision support GUI 300 is shown. Clinical decision support GUI 300 may be displayed as a pop-up window, side panel, or other type of modification to a patient timeline GUI, such as patient timeline GUI 200 of FIG. 2 in response to user selection of a decision support element thereof (e.g., decision support element 216 of FIG. 2). The clinical decision support GUI 300 may be displayed by a display of a care provider device (e.g., care provider device 134 of FIG. 1) in communication with a computing device and/or patient information system (e.g., patient information system 100 of FIG. 1).

The clinical decision support GUI 300 may display one or more activities for consideration by a care provider. The one or more activities may be based on or otherwise included in guideline recommendations (e.g., treatment or care recommendations) for a patient and display of an activity item for a patient may be based on a decision tree algorithm of rule sets stored in a rules module or other memory of the computing device. The activity item (e.g., activity recommendation) may be a suggested care measure (e.g., a suggested future care measure) such as a future intervention, future screening, or future treatment measure, like a medication, procedure, screening test, imaging test, and the like. The guideline recommendation indicated by the guideline recommendation panel 306 may aid the care provider in decision making for treatment based on the patient's past medical history as displayed within the patient timeline GUI.

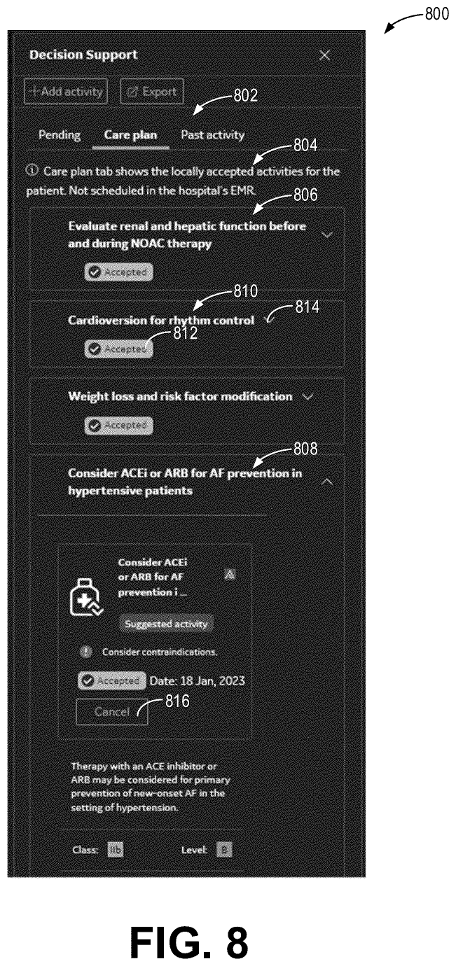

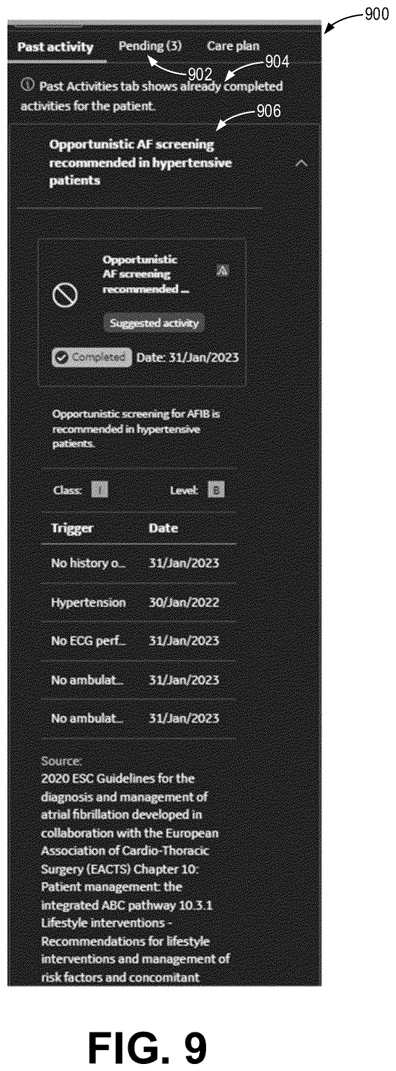

The clinical decision support GUI 300 may include a menu 302 with a plurality of headings (e.g., tabs), including pending, care plan, and past activity headings. The pending heading, when selected, may trigger display of suggested activities yet to be interacted with by the care provider in the clinical decision support GUI 300. The care plan heading, when selected, may trigger display of activities accepted by the care provider as well as information specific to the accepted activities in the clinical decision support GUI 300, as will be described with respect to FIG. 8. Activities in the care plan may be considered future treatments, interventions, screenings, etc. yet to be completed. The past activity heading, when selected, may trigger display of activities that have been completed and are therefore no longer included in the care plan, as will be described with respect to FIG. 9.

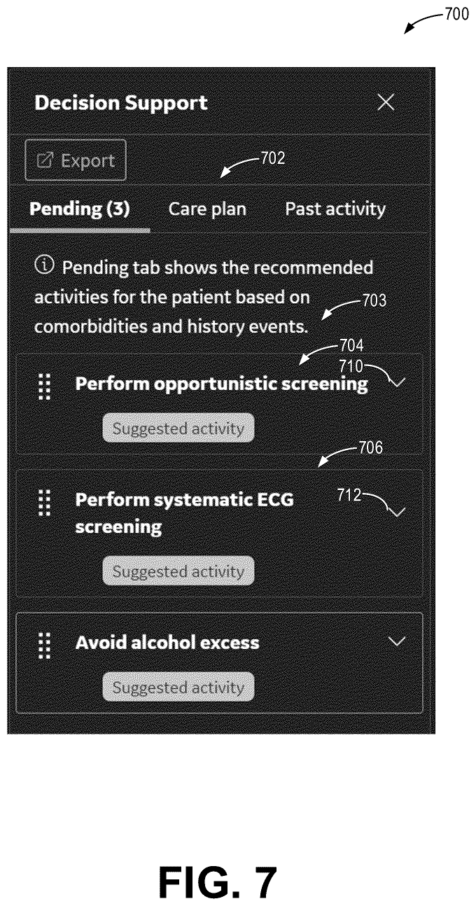

The clinical decision support GUI 300 as shown in FIG. 3 is depicted with the pending heading selected. When the pending heading is selected from the menu 302, clinical decision support GUI 300 may include an information panel 304 that includes a description of the selected heading and a guideline recommendation panel 306. The guideline recommendation panel 306 may include one or more pending activity items (e.g., activity recommendations). In some examples, each of the one or more pending activity items may be displayed at the same time in unexpanded states, displaying a limited amount of information for each activity, as will be further described with respect to FIG. 7. In other examples, as is depicted in FIG. 3, one of the one or more activity items may be displayed in an expanded state.

As an example, an activity item 307 in an expanded state that is displayed within the guideline recommendation panel 306 may be generated by a decision support module of a presentation system (e.g., decision support module 126 of presentation system 102 of FIG. 1). For example, the activity item 307 may be a recommendation for starting a medication, stopping a medication, proceeding with an intervention (e.g., a procedure), completing a screening, or other. The activity item 307 may be based on one of a plurality of guideline recommendations stored in memory of the presentation system 102. Each of the guideline recommendations may have defined or identified criteria and/or a rule set (e.g., a combination of triggers) that may be met by patient data in order for the activity item 307 to be displayed for the patient.

In some examples, the guideline recommendation panel 306 may include one or more selectable elements, including an accept element 308 and a cancel element 310. The accept element 308, when selected via user input, may trigger inclusion of a current or currently expanded activity item (e.g., activity item 307) in the care plan. The cancel element 310, when selected via user input, may trigger exclusion of the current activity item from the care plan. Selection of one of the plurality of selectable elements may also trigger display of a subsequent activity item or display of a notation indicating that no further pending activities are available. As an example, in an example in which three activity items are generated for a patient, a first activity may be accepted via user selection of the accept element 308, triggering expanded display of a second activity within the guideline recommendation panel 306. The second activity may then be rejected via user selection of the cancel element 310, triggering expanded display of a third activity item, which may be accepted or rejected in a similar fashion. In some examples, the accept element 308 and cancel element 310 may be displayed when an activity is in an expanded state. As such, acceptance or rejection of the activity may trigger a subsequent activity to be displayed in an expanded state. In another example, acceptance or rejection of the activity may trigger the guideline recommendation panel 306 to display each remaining pending activity item in an unexpanded state and the user may then select which of the remaining pending activities to expand and interact with.

In some examples, rejection of an activity item may trigger an additional pop-up window through which a user may indicate a reason for rejection. The patient information system may record in memory the reason for rejection. Stored reasons for rejection may affect future activity recommendations generated by the system for the patient or other patients.

In some examples, the guideline recommendation panel 306 may further include a description of a reasoning for the activity item 307 when the activity item 307 is in the expanded state. As an example, an activity suggesting addition of an angiotensin converting-enzyme (ACE) inhibitor or an angiotensin receptor blocker (ARB) to a patient's medications may include a description 312 stating in text that the suggested therapy may be considered for prevention of new-onset atrial fibrillation in the setting of hypertension. A guideline recommendation indicated to trigger display of the activity may include criteria such as hypertension (e.g., historical values of elevated blood pressure readings or historical medication lists including one or more anti-hypertensives) and no prior documented history of atrial fibrillation, though others are possible. The description 312 may be specific to the activity displayed in an expanded state within the guideline recommendation panel 306. For example, when a second activity is displayed in an expanded state in response to user input for a first activity, the description 312 may change to match the second activity.

The guideline recommendation panel 306 may further include a guideline class 314 and a level 316 of the activity specific to the activity item 307 being displayed within the guideline recommendation panel 306. The guideline class 314 may refer to a strength of the recommendation. For example, a class 1 guideline may be determined as strong, wherein the recommendation is considered effective/useful, while a class 2b may be considered weak, wherein the recommendation is considered potentially reasonable. Guideline classes may be predefined by a source from which the guideline recommendations were derived. The level 316 of the activity may be a confidence level or evidence level of the activity (e.g., activity item 307). The confidence or evidence level may be based on number and type of triggers that were met to identify the activity for display. The confidence levels may also be predefined and stored in memory of the computing device.

In some examples, the guideline recommendation panel 306 may also include a trigger list 318 and a corresponding date list 322. Each trigger in the trigger list 318 may be one of the triggers or criteria of a rule set that was met by the patient data in order to display the activity item 307 as a recommendation. Each date in the corresponding date list 322 may indicate a date of a corresponding trigger. For example, a trigger 320 of no atrial fibrillation in history may be found on a date 324.

While not shown in FIG. 3, the clinical decision support GUI 300 may further include, in some examples, a source description. The source description may indicate which clinical guideline recommendation source was used to identify a corresponding (e.g., a currently shown) activity item. For example, an item may have been identified based on ACC or ESC guidelines, as previously described.

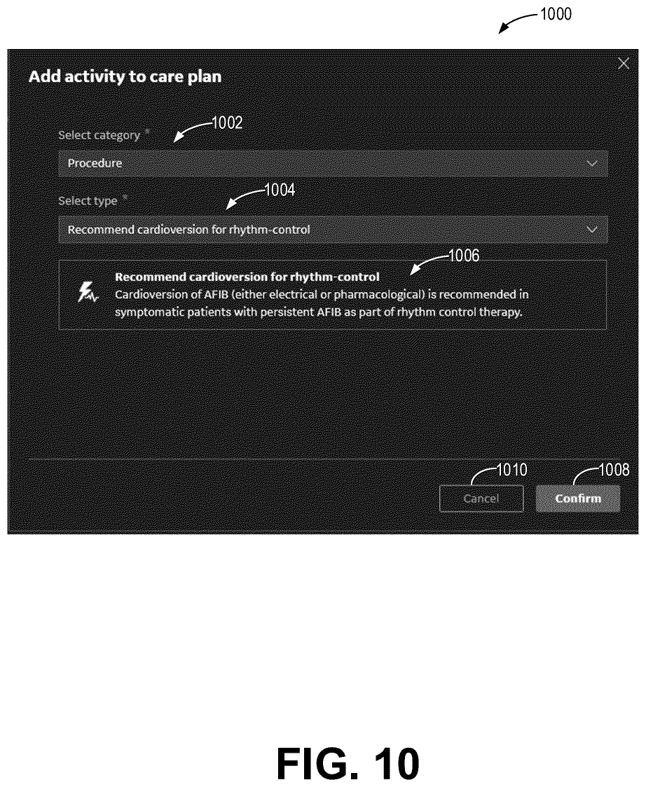

In some examples, the clinical decision support GUI 300 may also include an add activity element 330. When selected, the add activity element 330 may launch a pop-up window or additional panel within the clinical decision support GUI 300 in which the user may manually add an activity to a care plan, as will be further described with respect to FIG. 10.

As stated, the care plan heading of the menu 302 may be selected to display all of the accepted and manually added activities. An export button 332 may be selected by the user in order to generate an external file of the activities. The external file may be a portable document format (PDF) file or other type of file that is viewable by the care provider device. In some examples, the external file may include information of each of the activities included for the patient, including instructions for medications, descriptions of conditions, and more. In this way, patient adherence to the recommendations may be increased which may result in improved clinical outcomes.

Additionally, in some examples, the care plan may be exported to a connected EMR in order for accepted activities to be integrated into the EMR for action. For example, a care plan generated for a patient may include a new medication and a screening diagnostic exam. When exported back to the EMR, the new medication and the screening diagnostic exam may be added as orders for the patient, either automatically or via user selection of each care plan item.

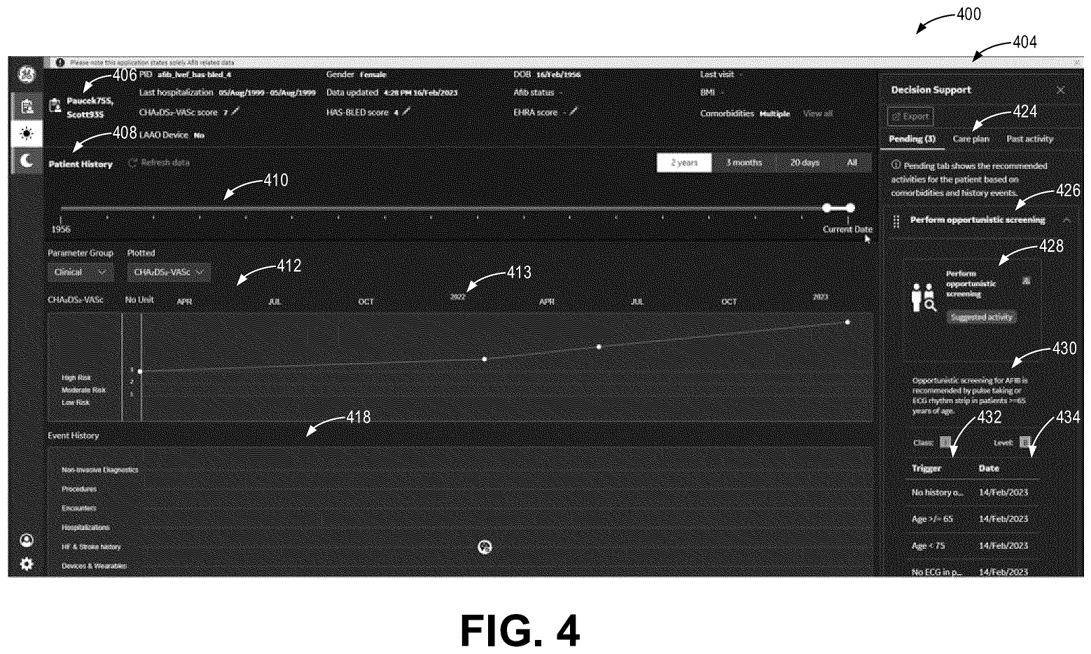

Turning now to FIG. 4, a second example of a patient timeline GUI 400 is shown. The patient timeline GUI 400 includes a clinical decision support GUI 404. Patient timeline GUI 400 may be displayed on a display device of a care provider device (e.g., care provider device 134 of FIG. 1) coupled to a patient information system (e.g., patient information system 100 of FIG. 1). The clinical decision support GUI 404 may be displayed as a side panel of the patient timeline GUI 400, as shown in FIG. 4, modifying a layout of the patient timeline GUI 400. Alternatively, the clinical decision support GUI 404 may be displayed as a separate pop-up window on top of the patient timeline GUI 400. In examples in which the clinical decision support GUI 404 is displayed as a separate pop-up window, a position of the clinical decision support GUI 404 may be changed via user input.

Panels and elements displayed within the patient timeline GUI 400, including a patient information panel 406, a patient history panel 408 that includes a time aligned graph 412 with a timeline 413, an event history panel 418, and other panels, and a timeline 410 may be similar to as described with reference to patient timeline GUI 200 of FIG. 2.

In a similar fashion, a menu 424 that includes tabs/headings for pending activities, a care plan, and past activities and a guideline recommendation panel 426 that includes one or more activity items 428 may be included in the clinical decision support GUI 404 similar to as described with reference to clinical decision support GUI 300 of FIG. 3. Expanded activity items within the guideline recommendation panel 426 may include an activity description 430 to provide textual information of a reasoning for a corresponding activity. The guideline recommendation panel 426 may further include a trigger list 432 and a date list 434. Each date within the date list 434 may corresponding to a trigger within trigger list 432, as described previously with respect to FIG. 3.

Referring now to FIG. 5, another example patient timeline GUI 500 is shown. Patient timeline GUI 500 specifically shows modification by user selection of a selectable element, as will be described. Patient timeline GUI 500 also depicts a medication panel 516 showing a longitudinal display of medications of different categories, as will be further explained. Patient timeline GUI 500 may be displayed on a display device of a care provider device (e.g., care provider device 134 of FIG. 1) coupled to a patient information system (e.g., patient information system 100 of FIG. 1) of a computing device.

Patient timeline GUI 500 may include an event history panel 502 of a patient history panel 501 for a patient with patient name 504. The event history panel 502 may include a plurality of event categories 506. Events corresponding to each of the event categories 506 may be time aligned (e.g., corresponding to a timeline not shown). As discussed previously, each event may be displayed as an event icon within the event history panel 502. For example, an event icon 510 may correspond to an event category 508. The event icon 510 may be displayed in a longitudinal manner, wherein event icons positioned leftwards of the event icon 510 may represent events having occurred prior to an event that the event icon 510 represents.

The event icon 510, and other event icons included in the event history panel 502 and/or other elements included in the patient timeline GUI, may be selectable elements that when selected or hovered over by an input device of the care provider device, launch a pop-up window. For example, event icon 510, when selected or hovered over, may launch a pop-up window 512. The pop-up window 512 may include additional information relating to the event represented by the event icon 510. The additional information may include, for example, a date of the event, an event type (e.g., a type of diagnostic exam, a type of encounter, a type of procedure, etc.), a result or exam finding, among others. Types of additional information included in the pop-up window may be determined by the category to which the event icon corresponds.

Additionally, in some examples, the pop-up window 512 may include a selectable link 514. The selectable link 514 may trigger display of one or more data repositories (e.g., one or more of the sources from which patient data was obtained when generating the patient timeline GUI 500). For example, a pop-up window corresponding to an ECG exam event may include a selectable link that when selected launches an ECG management system from which a corresponding ECG tracing may be displayed. In this way, data from multiple sources may be consolidated into a single interface wherein individual data points (e.g., history events) may be selected to display additional information or display full exams by linking to data repositories.

As noted, the patient timeline GUI 500 may also include the medication panel 516. As briefly discussed with respect to FIG. 2, the medication panel 516 may include one or more medication icons, such as medication icon 522. Each medication icon may correspond to a medication that the patient is currently taking or has previously taken. Each medication icon may correspond to one of a plurality of medication categories such as analgesics, non-steroidal anti-inflammatory analgesics, antiarrhythmics, anticoagulants, antiplatelets, etc. For example, medication icon 522 (e.g., ibuprofen) may correspond to medication category 518 (analgesics-non-steroidal anti-inflammatory). The medication icon 522 may be positioned along a duration line 524. The duration line 524 may begin at a start icon 520. The start icon 520 may indicate a start date for the medication to which the medication icon 522 corresponds. In examples in which the medication is a previous medication, the duration line 524 may also include an end icon indicating a date on which the medication was stopped. In examples in which the medication is a current medication, the duration line 524 may not include an end icon.

The medication icon 522 may include information such as name of the medication and dose of the medication. The medication icon 522 may be a selectable element that when selected or hovered over may launch display of a pop-up window. The pop-up window, similar to pop-up window 512 of the event history panel 502, may display additional information relating to the medication icon 522 such as medication name, start date, dose, frequency, etc. In this way, the user may visualize response to medication changes, for example changes in lab values, exam findings, etc. following start or stop of a medication.

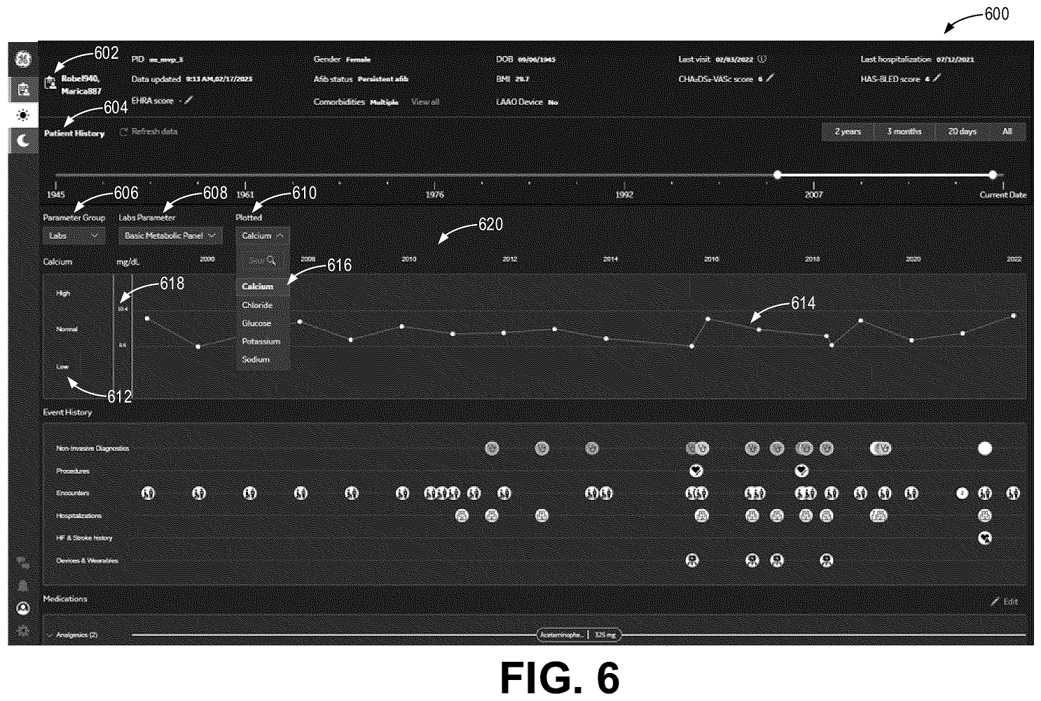

Referring now to FIG. 6, a third example of a patient timeline GUI 600 is shown. Patient timeline GUI 600 specifically depicts a patient history panel 604 for a specified patient 602 with additional drop-down menus. Patient timeline GUI 600 may be displayed on a display device of a care provider device (e.g., care provider device 134 of FIG. 1) coupled to a patient information system (e.g., patient information system 100 of FIG. 1) of a computing device.

In some examples, the patient timeline GUI 600 may include the patient history panel 604 that includes a time aligned graph 620 similar to time aligned graph 214 of FIG. 2. A parameter group drop-down menu 606 may include one or more parameter groups to be selected from. Based on the parameter group selected from the parameter group drop-down menu 606, an additional drop-down menu 608 may be displayed. For example, a lab parameter group may trigger display of the additional drop-down menu 608 that allows a user to select a lab parameter (e.g., a type of lab panel such as basic metabolic panel). Based on selection of the lab parameter, one or more plottable values may be displayed in a plotted drop-down menu 610. For example, for a lab parameter group and a lab parameter of basic metabolic panel, the values displayed in the plotted drop-down menu 610 may be lab tests 616 included in the basic metabolic panel (e.g., calcium, glucose, potassium, etc.). A selected lab test may be displayed in the time aligned graph 620, values for the selected lab test displayed as data points on line 614.

In examples in which the plotted value or parameter includes data of numbers (e.g., a lab value, algorithmic score, or the like), the time aligned graph 620 may include a value axis 618 as well as a category axis 612, both of which may be aligned as an ordinate of the graph. Categories shown in the category axis 612 may correspond to value ranges, for example a calcium over 10.4 mg/dL may be considered high, between 8.6 and 10.4 mg/dL may be considered normal, and below 8.6 mg/dL may be considered low. Corresponding values of those ranges may be shown in the value axis 618.

In this way, the user may be able to visualize trends over time as data points are plotted with respect to categories and values. Additionally, the user may correlate changes in data over time to events and/or medications included in an event history panel and/or medical panel to evaluate response to treatment. For example, a down-trending potassium value at a particular time may be correlated with start of a diuretic medication. Being able to visualize responses to treatments may aid informed decision making.

Referring now to FIG. 7, a second example of a clinical decision support GUI 700 is shown. Clinical decision support GUI 700 may be displayed in response to user interaction with a patient timeline GUI, such as user selection of a decision support element. Clinical decision support GUI 700 may be displayed on a display device of a care provider device (e.g., care provider device 134 of FIG. 1) coupled to a patient information system (e.g., patient information system 100 of FIG. 1). The clinical decision support GUI 700 is shown in FIG. 7 with multiple activity items 703. A menu 702 may be included in the clinical decision support GUI to toggle between pending activities, a care plan, and past activities (e.g., completed activities), as previously described.

In some examples, each of the multiple activity items 703 may be displayed in an unexpanded state, as is depicted in FIG. 7. Each of the activity items may include an expansion button. For example, a first activity item 704 may include a first expansion button 710 and a second activity item 706 may include a second expansion button 712. When selected via user input, the first expansion button 710 may trigger expansion of the first activity item 704. When unexpanded, the first activity item 704 may display limited information regarding a corresponding activity, for example a title of the activity without further description. Once expanded in response to selection of the first expansion button 710, the first activity item 704 may display additional information such as a description of a reasoning, triggers, and action elements (e.g., accept or cancel elements), as previously described with respect to FIG. 3. In this way, the user may visualize all activity items in unexpanded states and decide which to interact with, via user input to expand and accept or cancel, in a chosen order.

Turning now to FIG. 8, a third example of a clinical decision support GUI 800 is shown. While the clinical decision support GUIs 300, 404, and 700 are depicted with a pending heading selected and displaying pending activity items, the clinical decision support GUI 800 is shown in FIG. 8 with a care plan heading selected. Clinical decision support GUI 800 may be displayed on a display device of a care provider device (e.g., care provider device 134 of FIG. 1) coupled to a patient information system (e.g., patient information system 100 of FIG. 1) of a computing device.

In some examples as previously described, the clinical decision support GUI 800 may include a menu 802 via which a user may toggle between a pending activities tab, a care plan tab, and a past activities tab. In examples in which the care plan tab is selected, a plurality of accepted activities 806 may be displayed within the clinical decision support GUI 800. A description 804 may textually indicate what types of information are displayed in the care plan.

Each of the accepted activities 806 may include a title 810 and an icon 812 indicating a status (e.g., accepted). Each of the accepted activities 806 may further include an expansion button 814. When selected via user input, the expansion button 814 expands information displayed for a corresponding accepted activity. For example, accepted activity 808 is shown in an expanded state. Additional information such as description of reasoning for the activity, a date of acceptance, guideline class, level of activity, and triggers met to recommend the activity may be displayed as well similar to as previously described. Additionally, the accepted activity 808, in the expanded state, may include a cancel element 816 that, when selected, may remove the accepted activity 808 from the care plan. In this way, the user may be able to easily visualize all accepted activities within the care plan and find additional information about each accepted activity via user input with the clinical decision support GUI 800.

FIG. 9 depicts a fourth example of a clinical decision support GUI 900. Clinical decision support GUI 900 may be displayed on a display device of a care provider device (e.g., care provider device 134 of FIG. 1) coupled to a patient information system (e.g., patient information system 100 of FIG. 1). In some examples, clinical decision support GUI 900 may be the same as clinical decision support GUI 800 of FIG. 8 with a different heading selected. The clinical decision support GUI 900 depicts a menu 902 toggled to a past activities heading. The clinical decision support GUI 900 includes a description 904 of what information is displayed when the past activities heading is selected. Each activity displayed within the clinical decision support GUI 900 may be unexpanded or expanded, similar to as described with respect to FIG. 8.

In some examples, each activity displayed within the past activities heading is an activity that has been completed. For example, completed activity 906 shown in clinical decision support GUI 900 may be an activity that has been determined to be completed. Determination of completion may be time based, manually inputted, or based on patient data obtained from one or more sources. For example, an activity recommending starting a medication may be determined as completed when patient data obtained from the one or more sources includes the medication in a current medication list. As another example, an activity recommending opportunistic atrial fibrillation screening may be determined to be completed when an ECG has been performed within a specified timeframe of a current date (e.g., within 30 days). Once determined to be completed, activities may be removed from a care plan and may be displayed in the past activities tab.

In some examples, activities that have been completed may be suggested or recommended again after a certain time frame or if additional patient data is obtained that indicates the activity is to be recommended. As an example, a completed activity recommending an echocardiogram for a patient with congestive heart failure may be suggested again (e.g., included as a pending activity item) if a specified time frame (e.g., one year) has passed since a most recent echocardiogram. In other examples, activities that have been completed may not be suggested or recommended again if the activities are only relevant one time.

Referring to FIG. 10, an example of an add activity pop-up window 1000 is shown. Add activity pop-up window 1000 may be displayed on a display device of a care provider device (e.g., care provider device 134 of FIG. 1) coupled to a patient information system (e.g., patient information system 100 of FIG. 1). Add activity pop-up window 1000 may be displayed in response to user selection of an add activity element within a clinical decision support GUI, such as add activity element 330 of FIG. 3. In some examples, the add activity pop-up window 1000 may be displayed as a modification to a patient timeline GUI.

Add activity pop-up window 1000 may include one or more drop-down menus. For example, a select category menu 1002 and a select type menu 1004 may be drop-down menus that when selected via user input, trigger display of a list of available options. The select category menu 1002 may include options for categories of activities, which may include procedures, medications, lifestyle measures, screenings, and more. The select type menu 1004 may display options (e.g., available activity recommendations) specific to the category selected from the select category menu 1002. For example, type options for a procedure category may include cardioversion, ablation, implantable cardioverter defibrillator, and the like. In other examples, additional drop-down menus may be included.

The add activity pop-up window 1000 may further include a title/description 1006 of the selected activity type. For example, when a cardioversion is selected as a type of procedure, the title/description 1006 may display a title of the activity item as “cardioversion for rhythm-control”. The description may include a reasoning for the recommendation as derived from one or more guideline recommendation sources (e.g., ACC and/or ESC) as previously described.

The add activity pop-up window 1000 may include one or more selectable elements, such as a confirmation element 1008 and a cancel element 1010. Selection of the confirmation element 1008 may trigger inclusion of the selected activity item in a care plan displayed by the clinical decision support GUI to which the add activity pop-up window 1000 corresponds. Selection of the cancel element 1010 may trigger cancellation of any selections made within the add activity pop-up window 1000 and, in some examples, closure of the window.

In this way, a user may choose from relevant and available activity item options for a patient in the event a recommendation does not appear as a suggestion in the pending heading of the clinical decision support GUI. The add activity pop-up window may allow for customization of the care plan, thereby increasing efficiency and accuracy.

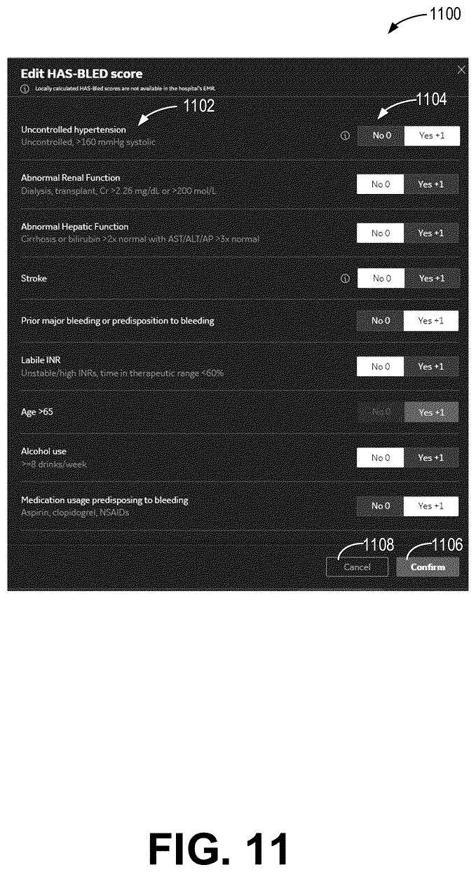

FIG. 11 depicts an example of an algorithmic score pop-up window 1100. The algorithmic score pop-up window 1100 may be an example of a pop-up window displayed in response to user selection of an element within a patient information panel of a patient timeline GUI (e.g., patient information panel 202 of patient timeline GUI 200 of FIG. 2). Other pop-up windows available from the patient information panel may include a comorbidities pop-up window, among others. The algorithmic score pop-up window 1100 may be displayed on a display device of a care provider device (e.g., care provider device 134 of FIG. 1) coupled to a patient information system (e.g., patient information system 100 of FIG. 1).

The algorithmic score pop-up window 1100 may include a parameter list 1102 that includes each parameter included in a corresponding algorithm and a score list 1104. Each score in the score list 1104 may correspond to a parameter in the parameter list 1102. For example, the algorithmic score pop-up window 1100 depicted in FIG. 11 is a HAS-BLED score. Parameters included in the parameter list 1102 for the HAS-BLED score may include uncontrolled hypertension, abnormal renal function, abnormal hepatic function, stroke, prior major bleeding or predisposition to bleeding, labile INR, age>65, alcohol use, and medication usage predisposing to bleeding. Each corresponding score in the score list 1104 may be either a no or a yes, wherein a no adds zero the resultant algorithm score and a yes adds one. Other algorithms may have different scoring systems. User input to each score within the score list 1104 may indicate a score for a corresponding parameter. Additionally, in some examples, parameters may include a descriptor of a positive score (e.g., a yes answer) to aid the user during information input.

In some examples, a selected score in the score list 1104 may be preset by the patient information system based on patient data acquired from the one or more sources. For example, patient data may indicate that a patient has abnormal renal function based on lab values showing elevated creatinine and/or low glomerular filtration rate (GFR) and as a result the corresponding score for that parameter may be set to a score of yes by the system. The user may make modifications or edits to preset scores for parameters if a care provider's determination is made that differs from the system's determination.

The algorithmic score pop-up window 1100 may also include a plurality of selectable elements, such as a confirmation element 1106 and a cancel element 1108. Selection of the confirmation element 1106 may trigger display of the overall algorithm score within the patient information panel of the patient timeline GUI from which the algorithmic score pop-up window 1100 was accessed. Selection of the cancel element 1010 may trigger cancellation of any selections made within the algorithmic score pop-up window 1100 and closure of the window. Cancellation of any selections may revert a score back to a prior determined score (e.g., a system determined score or prior user determined score).

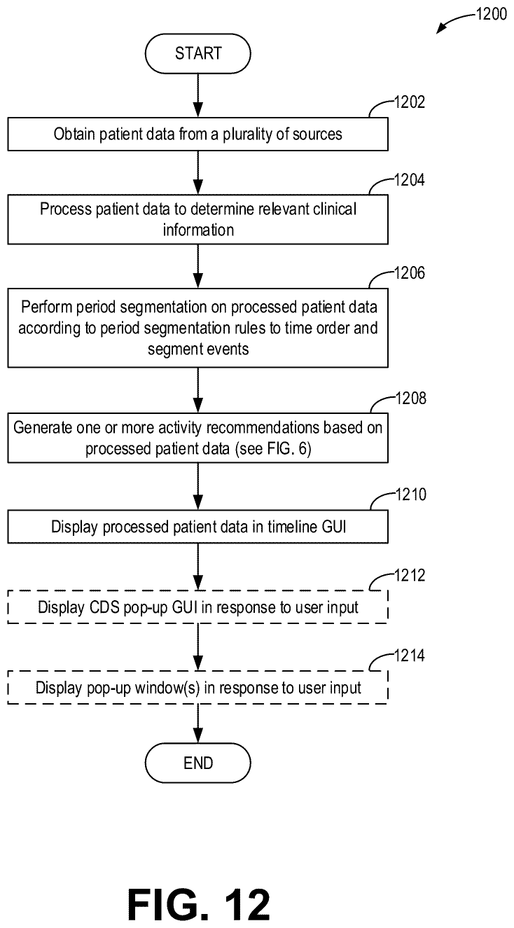

Turning now to FIG. 12, an example method 1200 for generating a timeline and one or more activities for a patient is shown. Method 1200 may be carried out according to instructions stored in memory of a computing device (e.g., memory 130 of presentation system 102 of FIG. 1), which may be executed by a processor of the computing device (e.g., processor(s) 132). While method 1200 is described specific to one patient, at least portions of method 1200 may be performed simultaneously for a plurality of patients.

At 1202, patient data from a plurality of sources is obtained for the patient. The patient data may be obtained from EMR databases, PACS, HIS/RIS/CIS, pathology systems, and more, as described previously with respect to FIG. 1. The patient data may include information of prior hospitalizations, pathology results, imaging exam findings, laboratory values, vital signs, medications, diagnosed conditions, and more. The patient data may include dates corresponding to each datum. The patient data may include all available data from each of the plurality of sources specific to the patient. The patient data may be obtained in response to a request (e.g., a user input or selection) to display a timeline of patient information/past medical history or automatically in response to an application being launched. For example, when viewing an EMR for the patient, a user may select a link displayed as part of the EMR interface in order to initiate generation of the timeline for the patient.

At 1204, the patient data is processed to determine relevant clinical information. A desired clinical setting may be introduced/defined to determine relevancy. In some examples, the desired clinical setting may be defined by user selection of a clinical setting from a list of possible clinical settings when launching the presentation system. For example, a link within an EMR that launches the patient timeline may include a drop-down menu through which the user may select a desired clinical setting. As another example, the patient timeline system, as a standalone application, may include an initial start page with a drop-down menu through which the user may select a desired clinical setting. In other examples, the desired clinical setting may be defined automatically based on an EMR from which the patient timeline was launched. For example, launching the patient timeline from an ECG management system may automatically define the desired clinical setting as cardiology.

In some examples, as is shown in FIGS. 2-11, the timeline and the one or more activities ultimately generated/recommended may be aimed at cardiology and, as such, data of the patient data irrelevant to cardiology may be processed or filtered out of the patient data. In this way, irrelevant information may be omitted from the timeline and activities recommended may be tuned to the desired clinical setting. In some examples, determination of relevancy may be based on one or more rules of a rules module of the patient information system (e.g., rules module 124 of FIG. 1), wherein types of data are predefined as relevant or irrelevant.

At 1206, period segmentation is performed on the processed patient data according to period segmentation rules to time order and segment events. The period segmentation rules may be included in the rules module of the patient information system. The patient data processed at 1204 may include multiple parameters of data of the patient's history, including clinical data, ECG data, and/or others, as described with respect to FIG. 2, as well as multiple types of data within each parameter. Data may be segmented into parameters and types; the data therefore being grouped. For example, data relevant to a condition such as atrial fibrillation may be segmented from data relevant to an imaging finding such as ejection fraction (indicating presence and severity of congestive heart failure). As a result, data may be presented in groups, wherein, for example, a time aligned graph displays information of a selected group and excludes data not included in the selected group. As described, each of the data may include a corresponding date (e.g., a date of diagnosis, a date of hospitalization, a date of procedure, etc.). Period segmentation may time order the patient data chronologically such that presentation of the data (e.g., display of the data within a patient timeline GUI) may be arranged chronologically.

At 1208, one or more activity items are generated based on the processed patient data. The processed patient data may be analyzed by a decision support module (e.g., decision support module 126 of FIG. 1) to determine which guideline recommendations have been met by the processed patient data (e.g., satisfying one or more rule sets). In some examples, indicated guideline recommendations based on the processed patient data may trigger generation of one or more activity items.

At 1210, the processed patient data is displayed in a patient timeline GUI. The patient timeline GUI (e.g., patient timeline GUI 200 of FIG. 2) may be outputted to a display of a care provider device (e.g., care provider device 134 of FIG. 1). The patient timeline GUI may include various panels presenting the patient data in a chronological manner. For example, a time aligned graph may be displayed indicating events for a chosen condition or other type of data and an event history panel may be displayed including time aligned event icons for a plurality of event types such as hospitalizations, procedures, and encounters. As described with reference to FIG. 2, each of the event icons or points on the time aligned graph may be selectable to display additional information.

Optionally at 1212, a clinical decision support pop-up GUI is displayed in response to user input. The clinical decision support pop-up GUI (e.g., clinical decision support GUI 300 of FIG. 3), may display the one or more activity items for review by the care provider. The patient timeline GUI may include a selectable element that when selected by the user, modifies the patient timeline GUI by launching the clinical decision support pop-up GUI.

Optionally at 1214, pop-up window(s) are displayed in response to user input. For example, user selection of a selectable element within a panel of the patient timeline GUI (e.g., a patient information panel, an event history panel, a time aligned graph, a medication panel, etc.) may launch display of a pop-up window. Depending on the type of element selected, the pop-up up window may display additional information relating to an event/icon or may display editable elements via which the user may modify algorithmic scores or comorbidities.

In this way, presenting relevant medical data obtained from a variety of disparate sources that may otherwise not be displayed in a single application for a patient in a chronological manner may aid a care provider in decision making and reduce cognitive overload by reducing amount of time spent searching for specific information. Additionally, by reducing amount of source applications that are individually accessed (e.g., in a launched state) by the computing device, processing demands of the computing device may be reduced.

Referring now to FIG. 13, an example method 1300 for clinical decision support is shown. Method 1300 may be carried out according to instructions, including instructions from a decision support module, stored in memory of a computing device (e.g., memory 130 of presentation system 102 of FIG. 1), which may be executed by a processor of the computing device (e.g., processor(s) 132).

At 1302, a patient timeline GUI is displayed of relevant patient data for a patient. As described with reference to method 1200 of FIG. 12, patient data may be obtained from a plurality of sources (e.g., one or more data repositories) and processed to exclude irrelevant data based on a desired clinical setting. The patient timeline GUI (e.g., patient timeline GUI 200 of FIG. 2) may display the relevant patient data in a plurality of time aligned panels and/or graphs. A time range may be specified by a user and only patient data within the time range may be displayed within the patient timeline GUI. The patient timeline GUI may include a plurality of selectable elements, as described with respect to FIG. 2, including a decision support element. The decision support element, when selected via user input, may trigger launch of a clinical decision support GUI (e.g., clinical decision support GUI 300 of FIG. 3).

At 1304, method 1300 judges whether user input to launch the clinical decision support GUI has been received. If user input has not been received (NO), method 1300 returns to 1302 to continue displaying the patient timeline GUI. If user input has been received (YES), method 1300 proceeds to 1306.

At 1306, the patient timeline GUI is modified to display the clinical decision support GUI with a first activity item in an expanded state. The clinical decision support GUI may be a pop-up GUI that is displayed either as an overlay on top of the patient timeline GUI or may modify a window of the patient timeline GUI to display the clinical decision support GUI as a side panel. In some examples, the placement of the clinical decision support GUI may be chosen by the user (e.g., via user modifiable user preferences or via user input to the clinical decision support GUI and/or patient timeline GUI).

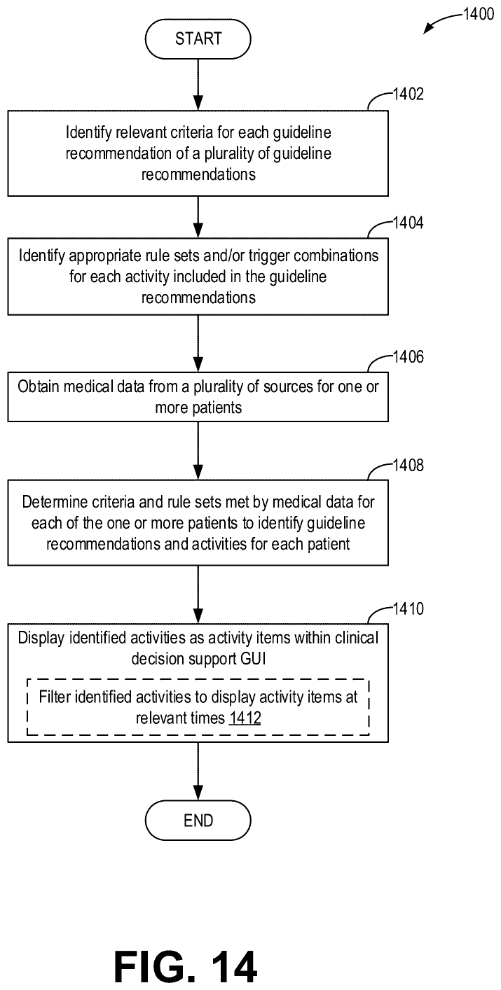

The clinical decision support GUI may display one or more activity items within a guideline recommendation panel, as described with respect to FIG. 3. Each of the activity items may be a suggested care measure (e.g., a treatment recommendation, medication recommendation, etc.) determined by the decision support module, as will be described below with reference to FIG. 14. The clinical decision support GUI further includes a plurality of selectable elements, including an accept element and a cancel element, for a currently expanded activity item. The accept element, when selected via user input, triggers acceptance of the activity item currently displayed in an expanded state. Accepted activity items may be added to a care plan. The cancel element, when selected via user input, triggers rejection of the activity item currently displayed in the expanded state. Rejected activity items may not be added to the care plan. Selection of either the accept element or the cancel element for a first activity item may trigger expansion (e.g., displayed in the expanded state) of a second recommendation if the one or more activity items includes more than one activity item.

The clinical decision support GUI may include the care plan, which displays each of the accepted activity items and in some examples additional information about each of the accepted activity items, and a past activity tab, which displays each completed activity item. The user may toggle between display of a pending tab (that includes the guideline recommendation panel), the care plan, and the past activities panel via a menu, as previously described.

Additionally, the user may manually add activities to the care plan. The clinical decision support GUI may include an add activity button that when selected launches a pop-up window with the clinical decision support GUI in which the user may add an activity not recommended by the decision support module, as described with reference to FIG. 3. Manually added activities may be included in the care plan similar to accepted activity items.

At 1308, method 1300 judges whether the first activity item has been accepted or rejected based on user input to the clinical decision support GUI. If user input has been received to accept the activity item, for example via user selection of the accept element, method 1300 proceeds to 1310. If user input has been received to reject the activity item, for example via user selection of the cancel element, method 1300 proceeds to 1312.

At 1310, the first activity item is added to the care plan following acceptance. The care plan, as described above, includes each of the accepted activity items and each of the manually added activities. The care plan may be a plan for the patient's treatment/care including added medications, stopped medications, recommended interventions, and the like.

At 1312, the clinical decision support GUI is displayed with a second activity item in the expanded state. The clinical decision support GUI may display the second activity item in response to user input in regards to the first activity item, for example either an acceptance or a rejection of the first activity item. In alternative examples in which only one activity item is generated by the decision support module for a patient, user input indicating acceptance or rejection of the activity item may trigger display of a notation that no further pending activities are available.

At 1314, method 1300 judges whether the second activity item has been accepted or rejected based on user input to the clinical decision support GUI. If user input has been received to accept the activity item, for example via user selection of the accept element, method 1300 proceeds to 1316. If user input has been received to reject the activity item, for example via user selection of the cancel element, method 1300 proceeds to 1318.

At 1316, the second activity item is added to the care plan. Prior to adding the second activity, the care plan may include the first activity item in examples in which the first activity item has been accepted. The care plan may also include any other accepted activity items as well as manually added activities, as described above. Following 1316, the care plan may include the second activity item in addition to activities previously in the care plan.

At 1318, user input is received to export the accepted recommendations. The clinical decision support GUI may include an export button or element, such as export button 332 of FIG. 3, that when selected triggers exportation of the care plan as an external file. User selection of the export button or element may be made from any of the tabs (e.g., headings) of the clinical decision support GUI, including a pending activities tab, the care plan tab, and/or the past activity tab.

In some examples, additional activity items may be generated by the decision support module. In such examples, in response to acceptance or rejection of the second activity item, subsequent activity items may be displayed, such as a third activity item. Further, in some examples, user input may be received to export the care plan prior to acceptance or rejection of all the activity items. For example, in an example in which three activity items are generated by the decision support module, the care plan may be exported following acceptance of the first and second activity items but prior to user selection of acceptance or rejection of the third activity item.

At 1320, a report of the accepted recommendations (e.g., from the care plan) is exported as a PDF file. In some examples, the type of file may be different than PDF, for example an XML paper specification (XPS) format or other format that the computing device may read and render in order for the care provider to take action with the file/document (e.g., print the document, email the file, etc.).

In some examples, the report of the accepted recommendations may include both a list of the accepted recommendations as well as information specific to each recommendation. For example, a report including an activity item for starting a new medication may include information regarding dosage, dosing frequency and duration, reasons for starting the medication including a description of a corresponding condition, pharmacy information, as well as instructions for taking the medication or lifestyle recommendations to accompany the medication.

Turning now to FIG. 14, an example method for identifying activity items for display within a clinical decision support GUI is shown. In some examples, identification of activity items may occur in response to a user launching the clinical decision support GUI. In other examples, identification of activity items may occur in response to the user launching a patient timeline GUI. Determination/identification of activities for display may be based on decision trees defining triggers and rule sets, as will be described. Method 1400 may be carried out according to instructions from a decision support module stored in memory of a computing device (e.g., memory 130 of presentation system 102 of FIG. 1), which may be executed by a processor of the computing device (e.g., processor(s) 132).

At 1402, relevant criteria are identified for each guideline recommendation of a plurality of guideline recommendations. Guideline recommendations may be known to the system (e.g., sourced from, for example, ACC and/or ESC, and stored in memory). In some examples, identification of criteria (e.g., triggers) for each guideline recommendation may be automatic based on the known clinical guidelines. In other examples, identification of criteria for each guideline may be configured manually by a clinical expert or care provider and inputted into the system, as described with reference to FIG. 1. The criteria for each guideline recommendation may be data obtainable for a patient from one or more sources (e.g., past medical history information including prior diagnoses, prior imaging/pathology/ECG findings, prior laboratory results or vital signs, among others). Each trigger may be defined based on a procedure code stored in the memory and thus determination of whether a particular trigger has been met may be based on a procedure code within patient information (e.g., an EMR) matching a procedure code stored in memory as part of the criteria for guideline recommendations. As described above, each guideline recommendation may include one or more activities that may be recommended if the guideline recommendation is indicated.

At 1404, appropriate rule sets and/or trigger combinations are identified for each activity of a set of activities included in the guideline recommendations. As noted, the criteria for each guideline recommendation may be a relatively large group of triggers possible to be met for a guideline recommendation to be indicated. Rule sets (e.g., trigger combinations) may be determined for each activity for relevant activities to be recommended for the patient. In this way, irrelevant or erroneous activity suggestions may be avoided. The rule sets and criteria for the guideline recommendations and activities may define one or more decision trees that are stored in memory.

For example, patient data may satisfy one trigger of a set of criteria for a guideline recommendation but not the other triggers in the set. Rule sets determined and included in a rules module of the system may dictate a combination of triggers that may be satisfied by the patient data in order for an activity to be indicated. Thus, the patient data does not indicate that the criteria for the guideline recommendation have been satisfied because only one trigger (of more than one triggers of a rule set) has been satisfied. In this way, while patient data may include triggers for one or more guideline recommendations, with determination of appropriate rule sets of the set of activities and which of the rule sets the patient data satisfies, only the relevant activities will be displayed as activity items for the patient. Rule sets may be stored in memory as decision trees and a decision tree algorithm may be performed by the decision support module to determine which rule sets are met. In some examples, activities may have individual decision trees. In other examples, dependent upon the patient data obtained, larger decision trees may lead to multiple recommended activities.

At 1406, medical data from a plurality of sources for one or more patients is obtained. The patient data may be obtained from EMR databases, an ECG management system, PACS, HIS/RIS/CIS, pathology systems, and more, as described previously with respect to FIG. 1. The medical data may include information of prior hospitalizations, pathology results, imaging exam findings, laboratory values, vital signs, medications, diagnosed conditions, ECG waveforms and/or findings, and more. The patient data may include dates corresponding to each datum. The patient data may include all available data from each of the plurality of sources and/or may be processed to filter out irrelevant data based on a desired setting (e.g., retaining data related to cardiology while filtering out other data). The patient data may be obtained in response to a request (e.g., a user input or selection) to display a timeline of patient information/past medical history or automatically in response to an application being launched.

At 1408, criteria and rule set(s) met by medical data for each of the one or more patients are determined to identify guideline recommendations and activities indicated for each patient. As described, each guideline recommendations may have identified criteria and rule sets that may be met in order for the activities to be indicated for a patient. Medical data for each patient may be analyzed to identify criteria (e.g., triggers), for example, based on matching procedure codes as described with respect to FIG. 1,. The identified criteria may then be analyzed to determine whether rule sets are met for each activity. If a rule set for an activity is met by medical data for a patient, the activity may be indicated for the patient and displayed as an activity item in a user interface. In this way, the user may be presented with clinically-based guidance that may increase efficiency and accuracy of clinical decisions, thereby improving patient care and clinical outcomes.

At 1410, identified activities are displayed as activity items within a clinical decision support GUI (e.g., the clinical decision support GUI 300 of FIG. 3). As described, the clinical decision support GUI may be displayed on top of or otherwise as part of a patient timeline GUI (e.g., patient timeline GUI 200 of FIG. 2) that displays the medical data obtained from the plurality of sources in a time arranged manner. The clinical decision support GUI may display each identified activity that is to either be accepted or rejected by a user via user input to the clinical decision support GUI, as previously described.

In some examples, the identified activities are filtered to display activity items at relevant times, as noted at 1412. For example, an activity may be identified for suggestion for a patient, but the activity may have already been suggested, accepted, and completed for the patient. In some examples, the activity may not be displayed as an activity item until a specified time frame has passed. In other examples, the activity may not be displayed as an activity item if it is determined to be an activity that may be suggested only once.

The technical effect of presenting patient data in a patient timeline GUI as disclosed herein is that user/clinician time spent searching for relevant patient data may be reduced and inaccurate care plans due to missing or overlooked patient data may be mitigated, while also decreasing network traffic and/or reducing processing demands by reducing the number of user interactions with various interfaces (e.g., to look up and visualize the patient data). When the patient timeline GUI and/or the clinical decision support GUI are displayed and when the additional display elements (e.g., the various pop-up windows described herein) are displayed, the one or more data repositories (e.g., EMRs, PACS, ECG management systems, etc.) may exist in an un-launched state (e.g., not displayed and not currently accessed by the display device.) In this way, the data from the data repositories may be used to generate medical history event elements (e.g., event icons of panels, time-aligned graphs, etc.) and activity items. The patient data may be obtained from the data repositories (e.g., lab values, imaging results, prior diagnoses, medication lists, etc.) and used to populate the panels and time aligned graphs displayed without the user having to access the data repositories themselves (e.g., in a separate window). In doing so, the patient timeline GUI as disclosed herein may present a limited set of information (e.g., the medical history event elements of panels and graphs) to the users/care providers in order to increase efficiency of the users' interaction with the available data as users are not forced to sift through multiple separate EMRs, data feeds, data files, etc., to identify and then aggregate the needed information.

The systems, methods, and graphical user interfaces provided herein may improve the accuracy and timeliness of clinical decision making for care providers in high-demand, high-resource settings (e.g., high-volume medical facility units such as cardiology units). This may be particularly useful during high-demand situations or when patient data spans multiple years and multiple EMRs, such as is the case for chronic cardiology conditions that demand long term monitoring and treatment. The time it would take to individually collect data from multiple data repositories, aggregate and analyze the collected data, and visualize the data using standard methods may take inordinate amounts of time due to data volume. By establishing a system that presents relevant data longitudinally and makes suggestions for care measures in the form of activity items, time spent searching for relevant data may be reduced and inaccurate care plans due to missing information may be mitigated. Mitigation of inaccurate care plans may thereby result in cost reductions for medical facilities and patients. Further, reducing generation of inaccurate care plans and therefore reducing demand for redoing care plan may increase efficiency and reduce processing power of the computing devices.

The suggested care measures generated by the systems and methods herein may be evidence-based (e.g., sourced from known clinical knowledge, for example from the ACC and/or ESC), which may in turn increase standardization of care by incorporating evidence-based guidelines that impact treatment decisions. The relevant data obtained from the plurality of data repositories may enable personalized care delivery for favorable patient outcomes. By incorporating the use of patient care guidelines, the system may be able to consume existing medical data and suggest care plans based off algorithm decision trees. The proposed care plans may be accepted or replaced with clinical override (e.g., via user interaction with the interface). The care plan may be exported and made shareable by coordinated care teams as well as shared with the patient for the purpose of optimal patient and operational outcomes.

In contrast, in prior systems when a care provider attempted to determine a care plan for a patient with a substantial cardiac history spanning multiple hospitals and multiple EMRs over the course of many years, errors and/or delays could be made due to delays in individual care providers obtaining all necessary parameters for evaluating a patient's current condition and past medical history. For example, a patient's medical history may indicate a predisposition to bleeding, but that information may be buried amongst a vast amount of other information. If that information missed by a care provider manually searching the patient's vast medical history, a particular medication, such as an anticoagulant, may be added in error. The longitudinal patient history timeline system and the generated GUIs therein addresses this problem by sampling all available data repositories for relevant patient history data, presenting that data visually in a longitudinal (e.g., chronologic) manner, and generating one or more suggested care measures based on known clinical guidelines (e.g., in the form of activity items). The disclosure provides a specific way of improving the capability of the healthcare system, by providing one or more GUIs that display longitudinal patient history data and suggested care measures. The disclosure further provides a specific improvement to the way computers operate by aggregating data from multiple sources (e.g., EMRs, PACS, RIS, etc.) in one location, which may obviate the need for users to have to navigate through multiple sources, manually update information, and so forth, thereby increasing the efficiency of the operation of the computer for the user.

The patient timeline GUIs and clinical decision support GUIs described herein provide a specific manner of displaying a limited set of information to a user (e.g., medical history events as elements and suggested care measures as activity items), rather than using conventional user interface methods to display a generic index on a computer, requiring the user to step through multiple layers of menu options to access the desired data, or burying the desired data within all hospital data. Thus, the user experience with the computer may be improved and made more efficient.

Furthermore, by displaying a limited set of information via the patient timeline GUIs and clinical decision support GUIs as described herein, operation of the computing device(s) that collect and render the data for display may be improved by reducing the processing demands of the computing device(s), thereby increasing the efficiency of the computing device(s). For example, only certain patient data may be displayed based on a defined desired clinical setting, which results in a limited amount of the data that is received being processed for display, which may improve the efficiency of the computing device(s).

The longitudinal timeline and clinical decision support system, included as part of a medical information system as described herein, may provide for high speed data processing that allows for display of multiple data types within a single interface. Thus, via the disclosed longitudinal timeline and clinical decision support system, relational patient history data relevant to a desired clinical setting (e.g., cardiology) may be displayed in a manner that is easy to visually parse and act on in a reduced amount of time.

The disclosure also provides support for a computing device comprising a display screen, the computing device being configured to display on the display screen a menu listing one or more electronic medical record (EMRs) of one or more patients, and additionally being configured to display on the display screen a patient timeline graphical user interface (GUI) accessible from the menu, wherein the patient timeline GUI displays, for each patient, patient data as longitudinal medical history event elements, the patient data obtained from the one or more EMRs, wherein each element of the longitudinal medical history event elements is selectable to launch a pop-up window with additional information relating to the selected element, wherein the computing device is additionally configured to generate one or more activities based on the patient data, and wherein the patient timeline GUI is displayed while the one or more EMRs are in an un-launched state. In a first example of the system, the patient data is segmented into a plurality of parameters and each of the plurality of parameters is further segmented into a plurality of event types. In a second example of the system, optionally including the first example, each of the plurality of event types are displayed in separate time aligned panels, each event within an event type being displayed as an event icon within one of the separate time aligned panels. In a third example of the system, optionally including one or both of the first and second examples, the event icon is one of the longitudinal medical history event elements and is selectable to launch the pop-up window with additional information relating to a corresponding event, the additional information including a date of the corresponding event, a category of the corresponding event, and/or a result of the corresponding event. In a fourth example of the system, optionally including one or more or each of the first through third examples, the patient timeline GUI includes a decision support element, the decision support element being selectable to launch a clinical decision support GUI, the clinical decision support GUI being a modification to the patient timeline GUI wherein the clinical decision support GUI is displayed as a side panel of the patient timeline GUI. In a fifth example of the system, optionally including one or more or each of the first through fourth examples, the clinical decision support GUI comprises a plurality of selectable elements, including an accept element and a cancel element, wherein the accept element, when selected for a first activity, triggers inclusion of the first activity to a care plan and the cancel element, when selected for a second activity, triggers exclusion of the second activity from the care plan. In a sixth example of the system, optionally including one or more or each of the first through fifth examples, the clinical decision support GUI displays, for each patient, the one or more activities, the one or more activities being assigned based on the patient data. In a seventh example of the system, optionally including one or more or each of the first through sixth examples, the one or more activities comprise one or more of a recommendation for future care, a recommendation for future treatment, a recommendation for future intervention, and a recommendation for future screening. In a eighth example of the system, optionally including one or more or each of the first through seventh examples, the one or more activities are assigned from a set of activities based on a plurality of guideline recommendations. In a ninth example of the system, optionally including one or more or each of the first through eighth examples, each of the plurality of guideline recommendations comprises one or more triggers of a plurality of triggers, each activity of the one or more activities associated with a rule set including one or more of the plurality of triggers.

The disclosure also provides support for a method for a longitudinal cardiology timeline and clinical decision support system, comprising: displaying a menu listing one or more options for retrieving data of one or more patients from a plurality of data repositories of a hospital, the plurality of data repositories including one or more electronic medical record (EMR) systems, displaying a patient timeline graphical user interface (GUI) that displays, for each patient, a plurality of elements indicating a plurality of history events determined from the retrieved data from the one or more EMR systems, the plurality of elements being arranged chronologically, and in response to selection of an element of the plurality of elements, modifying the patient timeline GUI to display a clinical decision support GUI that displays one or more activity items, wherein the patient timeline GUI is displayed while the one or more EMR systems are in an un-launched state. In a first example of the method, the one or more activity items are identified based on a set of rules applied to the retrieved data, the set of rules identifying one or more rule sets to be met in order to identify the one or more activity items, wherein each of the one or more rule sets includes one or more trigger combinations. In a second example of the method, optionally including the first example, the set of rules are based on a set of known clinical guidelines stored in memory of a computing device. In a third example of the method, optionally including one or both of the first and second examples, the one or more activity items comprise recommendations for future care for each patient. In a fourth example of the method, optionally including one or more or each of the first through third examples, the clinical decision support GUI comprises a plurality of selectable elements that, when selected, trigger inclusion or exclusion of a corresponding activity to a care plan, the care plan comprising a list of all included activities for a specified patient.

The disclosure also provides support for a longitudinal cardiology patient history timeline system, comprising: one or more processors, and memory storing instructions executable by the one or more processors to: output, for display on a display device, a patient timeline graphical user interface (GUI) that includes, for a patient, a plurality of panels indicating patient history events, where each panel is generated by applying a set of rules to a set of patient data obtained from an electronic medical record database, and display, within a modification of the patient timeline GUI, an activity recommendation indicating a suggested care measure based on the patient data. In a first example of the system, the instructions are executable to display, on the patient timeline GUI, one or more event icons, each of the one or more event icons being selectable to launch a pop-up window displaying additional information relating to an event represented by a selected event icon. In a second example of the system, optionally including the first example, the modification of the patient timeline GUI is a clinical decision support GUI, the clinical decision support GUI, when launched, being displayed as a side panel of the patient timeline GUI. In a third example of the system, optionally including one or both of the first and second examples, the clinical decision support GUI displays the activity recommendation and one or more selectable elements, the activity recommendation being added to a care plan via user selection of one of the one or more selectable elements. In a fourth example of the system, optionally including one or more or each of the first through third examples, the clinical decision support GUI includes an add activity element that when selected launches an add activity pop-up window, the add activity pop-up window displaying, via user input to one or more drop-down menus, a subset of available activity recommendations.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects. This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A computing device comprising a display screen, the computing device being configured to display on the display screen a menu listing one or more electronic medical record (EMRs) of one or more patients, and additionally being configured to display on the display screen a patient timeline graphical user interface (GUI) accessible from the menu, wherein the patient timeline GUI displays, for each patient, patient data as longitudinal medical history event elements, the patient data obtained from the one or more EMRs, wherein each element of the longitudinal medical history event elements is selectable to launch a pop-up window with additional information relating to the selected element, wherein the computing device is additionally configured to generate one or more activities based on the patient data, and wherein the patient timeline GUI is displayed while the one or more EMRs are in an un-launched state.

2. The computing device of claim 1, wherein the patient data is segmented into a plurality of parameters and each of the plurality of parameters is further segmented into a plurality of event types.

3. The computing device of claim 2, wherein each of the plurality of event types are displayed in separate time aligned panels, each event within an event type being displayed as an event icon within one of the separate time aligned panels.

4. The computing device of claim 3, wherein the event icon is one of the longitudinal medical history event elements and is selectable to launch the pop-up window with additional information relating to a corresponding event, the additional information including a date of the corresponding event, a category of the corresponding event, and/or a result of the corresponding event.

5. The computing device of claim 1, wherein the patient timeline GUI includes a decision support element, the decision support element being selectable to launch a clinical decision support GUI, the clinical decision support GUI being a modification to the patient timeline GUI wherein the clinical decision support GUI is displayed as a side panel of the patient timeline GUI.

6. The computing device of claim 5, wherein the clinical decision support GUI comprises a plurality of selectable elements, including an accept element and a cancel element, wherein the accept element, when selected for a first activity, triggers inclusion of the first activity to a care plan and the cancel element, when selected for a second activity, triggers exclusion of the second activity from the care plan.

7. The computing device of claim 5, wherein the clinical decision support GUI displays, for each patient, the one or more activities, the one or more activities being assigned based on the patient data.

8. The computing device of claim 1, wherein the one or more activities comprise one or more of a recommendation for future care, a recommendation for future treatment, a recommendation for future intervention, and a recommendation for future screening.

9. The computing device of claim 1, wherein the one or more activities are assigned from a set of activities based on a plurality of guideline recommendations.

10. The computing device of claim 9 wherein each of the plurality of guideline recommendations comprises one or more triggers of a plurality of triggers, each activity of the one or more activities associated with a rule set including one or more of the plurality of triggers.

11. A method for a longitudinal cardiology timeline and clinical decision support system, comprising:
displaying a menu listing one or more options for retrieving data of one or more patients from a plurality of data repositories of a hospital, the plurality of data repositories including one or more electronic medical record (EMR) systems;
displaying a patient timeline graphical user interface (GUI) that displays, for each patient, a plurality of elements indicating a plurality of history events determined from the retrieved data from the one or more EMR systems, the plurality of elements being arranged chronologically; and
in response to selection of an element of the plurality of elements, modifying the patient timeline GUI to display a clinical decision support GUI that displays one or more activity items, wherein the patient timeline GUI is displayed while the one or more EMR systems are in an un-launched state.

12. The method of claim 11, wherein the one or more activity items are identified based on a set of rules applied to the retrieved data, the set of rules identifying one or more rule sets to be met in order to identify the one or more activity items, wherein each of the one or more rule sets includes one or more trigger combinations.

13. The method of claim 12, wherein the set of rules are based on a set of known clinical guidelines stored in memory of a computing device.

14. The method of claim 11, wherein the one or more activity items comprise recommendations for future care for each patient.

15. The method of claim 11, wherein the clinical decision support GUI comprises a plurality of selectable elements that, when selected, trigger inclusion or exclusion of a corresponding activity to a care plan, the care plan comprising a list of all included activities for a specified patient.

16. A longitudinal cardiology patient history timeline system, comprising:
one or more processors; and
memory storing instructions executable by the one or more processors to:
output, for display on a display device, a patient timeline graphical user interface (GUI) that includes, for a patient, a plurality of panels indicating patient history events, where each panel is generated by applying a set of rules to a set of patient data obtained from an electronic medical record database; and
display, within a modification of the patient timeline GUI, an activity recommendation indicating a suggested care measure based on the patient data.

17. The longitudinal cardiology patient history timeline system of claim 16, wherein the instructions are executable to display, on the patient timeline GUI, one or more event icons, each of the one or more event icons being selectable to launch a pop-up window displaying additional information relating to an event represented by a selected event icon.

18. The longitudinal cardiology patient history timeline system of claim 16, wherein the modification of the patient timeline GUI is a clinical decision support GUI, the clinical decision support GUI, when launched, being displayed as a side panel of the patient timeline GUI.

19. The longitudinal cardiology patient history timeline system of claim 18, wherein the clinical decision support GUI displays the activity recommendation and one or more selectable elements, the activity recommendation being added to a care plan via user selection of one of the one or more selectable elements.

20. The longitudinal cardiology patient history timeline system of claim 18, wherein the clinical decision support GUI includes an add activity element that when selected launches an add activity pop-up window, the add activity pop-up window displaying, via user input to one or more drop-down menus, a subset of available activity recommendations.
